(19) United States
(12) Patent Application Publication
(10) Pub. No.: US 2022/0136070 A1
(43) Pub. Date: May 5, 2022

(54) METHODS AND SYSTEMS FOR CHARACTERIZING TUMOR RESPONSE TO IMMUNOTHERAPY USING AN IMMUNOGENIC PROFILE

(71) Applicant: OmniSeq, Inc., Buffalo, NY (US)

(72) Inventors: Sarabjot Pabla, Buffalo, NY (US); Jeffrey Conroy, Williamsville, NY (US)

(73) Assignee: OmniSeq, Inc., Buffalo, NY (US)

(21) Appl. No.: 17/452,873

(22) Filed: Oct. 29, 2021

Related U.S. Application Data
(60) Provisional application No. 63/107,906, filed on Oct. 30, 2020.

(51) Int. Cl. C12Q 1/6886

(52) U.S. Cl. CPC C12Q 1/6886; C12Q 2600/106; C12Q 2600/158; C12Q 2600/112; C12Q 2600/118

(57) ABSTRACT

A method for characterizing response of a tumor to immunotherapy, including: (i) obtaining tissue from the tumor; (ii) generating, from the obtained tissue, an immune gene expression dataset comprising gene expression data for a plurality of immune genes; (iii) calculating, from the immune gene expression dataset, an immunogenic signature score; (iv) identifying, based on the calculated immunogenic signature score, the tumor as strongly immunogenic, moderately immunogenic, or weakly immunogenic; and (v) predicting, based on the identification of the tumor as strongly immunogenic, moderately immunogenic, or weakly immunogenic, the response of the tumor to immunotherapy.

### CROSS-REFERENCE TO RELATED APPLICATION

The present application claims priority to and the benefit of U.S. Provisional Patent Application Ser. No. 63/107,906, filed on Oct. 30, 2021 and entitled “Methods and Systems for Characterizing Tumor Response to Immunotherapy Using an Immunogenic Profile,” the entire contents of which is hereby incorporated by reference in its entirety.

### FIELD OF THE DISCLOSURE

The present disclosure is directed generally to methods and systems for characterizing tumor response to immunotherapy.

### BACKGROUND

Since the approval of the first immune checkpoint inhibitor (ICI) for melanoma the landscape of cancer therapies has changed dramatically, combining biological response with genomics knowledge to change treatment paradigms and improve clinical outcomes. Immunotherapies have shown to significantly improve clinical endpoints such as progression free survival and overall survival in multiple cancer subtypes compared to chemotherapy alone. Despite the tremendous efficacy of ICIs in some patients, other patients fail to respond to therapy, while others can develop severe autoimmune toxicity. To maximize treatment benefit and develop personalized therapeutic strategies, genomic and immune biomarkers such as PD-L1 and tumor mutational burden (TMB, a quantitative measure of the total number of gene mutations inside cancer tumor cells) are utilized to guide therapeutic decisions based on tumor subtype. Although biomarker analyses regularly guide treatment decisions in standard of care clinical settings, single biomarkers alone are insufficient to adequately predict therapeutic response in some patients. As a result, there is increased demand for the development of predictive assays which consider the multitude of networks and cellular phenotypes that complicate the immune tumor microenvironment (TME).

Proximity between tumor cells and immune cells is essential, though not entirely sufficient, for immunotherapy efficacy as tumors can avoid destruction by immune escape mechanisms such as downregulation of antigens, recruitment of immune suppressors, and upregulation of receptors that downregulate tumor-infiltrating lymphocytes (TILs). It is well known that the success of ICIs depends upon the mobilization of the immune system within the TME where cancer cells interact with stromal cells. Therefore, the development of a biomarker detection modality inclusive of both cell proliferation and inflammation biomarkers is necessary to improve patient management.

In a recent study, an RNA-seq gene expression profile (GEP) consisting of IFN-gamma genes, chemokine expression, cytotoxic activity and immune resistance genes, along with PD-L1 and TMB, was analyzed. While the T cell-inflamed GEP signatures correlated with clinical benefit for ICI therapy, the addition of all the gene profiles in the GEP did not exhibit sufficient sensitivity to characterize the clinical benefit. Thus, although tumor profiles have previously been generated and analyzed in order to characterize the tumor's predicted response to immunotherapy such as ICI therapy, these previous methods exhibit low sensitivity and insufficient predictive power.

### SUMMARY OF THE DISCLOSURE

There is therefore a continued need for highly sensitive and effective methods and systems to characterize tumor response to immunotherapy. Various embodiments and implementations herein are directed to methods for generating and analyzing a tumor profile. The methods utilize combinations of immune and neoplastic influences responsible for response to ICI, beyond a comprehensive immunogenic signature. The method utilizes tissue obtained from a tumor, which is used to generate an immune gene expression dataset comprising gene expression data for a plurality of immune genes. An immunogenic signature score is generated from the immune gene expression dataset, and the tumor is categorized as strongly immunogenic, moderately immunogenic, or weakly immunogenic based on the immunogenic signature score. The response of the tumor to immunotherapy can then be predicted based on the identification of the tumor as strongly immunogenic, moderately immunogenic, or weakly immunogenic.

Generally, in one aspect, a method for characterizing response of a tumor to immunotherapy is provided. The method includes: (i) obtaining tissue from the tumor; (ii) generating, from the obtained tissue, an immune gene expression dataset comprising gene expression data for a plurality of immune genes; (iii) calculating, from the immune gene expression dataset, an immunogenic signature score; (iv) identifying, based on the calculated immunogenic signature score, the tumor as strongly immunogenic, moderately immunogenic, or weakly immunogenic; and (v) predicting, based on the identification of the tumor as strongly immunogenic, moderately immunogenic, or weakly immunogenic, the response of the tumor to immunotherapy.

According to an embodiment, the plurality of immune genes comprises at least the 161 genes of Table 4. According to an embodiment, the plurality of immune genes comprises only the 161 genes of Table 4. According to an embodiment, the plurality of immune genes comprises a subset of the 161 genes of Table 4.

According to an embodiment, the immunogenic signature score comprises a mean expression rank for the gene expression data for the plurality of immune genes.

According to an embodiment, the method further includes: generating, from the obtained tissue, a cell proliferation gene expression dataset comprising gene expression data for a plurality of cell proliferation genes; calculating, from the cell proliferation gene expression dataset, a cell proliferation score; and identifying, based on the calculated cell proliferation score, the tumor as highly proliferative, moderately proliferative, or poorly proliferative; wherein predicting the response of the tumor to immunotherapy is further based on the identification of the tumor as highly proliferative, moderately proliferative, or poorly proliferative.

According to an embodiment, the method further includes generating, from the obtained tissue, a PD-L1 expression profile by quantitative or qualitative measurement, wherein predicting the response of the tumor to immunotherapy is further based on the generated PD-L1 expression profile.

According to an embodiment, the method further includes generating, from the obtained tissue, a tumor mutational burden (TMB) profile, wherein the TMB profile comprises mutational burden information about a plurality of genes generated from DNA sequencing data; wherein predicting the response of the tumor to immunotherapy is further based on the generated TMB profile.

According to an embodiment, the gene expression data is generated by RNA sequencing.

According to an embodiment, the method further includes determining, using the predicted response of the tumor to immune checkpoint blockade therapy, a therapy for the tumor.

According to an embodiment, the tumor as is identified as strongly immunogenic when the calculated immunogenic signature score (IS) is equal to and/or greater than [Median IS]_{Borderline}+2 ×[Std. Dev. IS]_{Borderline}, wherein [Median IS]_{Borderline} is a median determined for a set of immunogenic signature scores calculated for a plurality of patients categorized as borderline inflamed, and [Std. Dev. IS]_{Borderline} is one standard deviation of the set of immunogenic signature scores calculated for the plurality of patients categorized as borderline inflamed.

According to an embodiment, the tumor as is identified as weakly immunogenic when the calculated immunogenic signature score (IS) is equal to and/or less than [Median IS]_{Noninflamed}+2×[Std. Dev. IS]_{Noninflamed}, wherein [Median IS]_{Noninflamed} is a median determined for a set of immunogenic signature scores calculated for a plurality of patients categorized as noninflamed, and [Std. Dev. IS]_{Noninflamed} is one standard deviation of the set of immunogenic signature scores calculated for the plurality of patients categorized as noninflamed.

According to an embodiment, the tumor is identified as moderately immunogenic when the calculated immunogenic signature score (IS) determined to be less than a strongly immunogenic score and greater than a weakly immunogenic score.

According to another aspect is a method for characterizing response of a tumor to immunotherapy. The method includes: (i) obtaining tissue from the tumor; (ii) generating, from the obtained tissue: (1) an immune gene expression dataset comprising gene expression data for a plurality of immune genes; (2) a PD-L1 expression profile; and (3) a tumor mutational burden (TMB) profile, wherein the TMB profile comprises mutational burden information about a plurality of genes generated from DNA sequencing data; (iii) calculating, from the immune gene expression dataset, an immunogenic signature score; (iv) identifying, based on the calculated immunogenic signature score, the tumor as strongly immunogenic, moderately immunogenic, or weakly immunogenic; and (v) predicting, based on: (1) the identification of the tumor as strongly immunogenic, moderately immunogenic, or weakly immunogenic; (2) the generated PD-L 1 expression profile; and (3) the generated TMB profile, the response of the tumor to immunotherapy.

According to an embodiment, the method further includes generating, from the obtained tissue, a cell proliferation gene expression dataset comprising gene expression data for a plurality of cell proliferation genes; calculating, from the cell proliferation gene expression dataset, a cell proliferation score; and identifying, based on the calculated cell proliferation score, the tumor as highly proliferative, moderately proliferative, or poorly proliferative; wherein predicting the response of the tumor to immunotherapy is further based on the identification of the tumor as highly proliferative, moderately proliferative, or poorly proliferative.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims.

FIG. 1A is a flowchart of a method for characterizing response of a tumor to immunotherapy, in accordance with an embodiment.

FIG. 1B is a flowchart of a method for characterizing response of a tumor to immunotherapy, in accordance with an embodiment.

FIG. 2 is a graph of immunogenic signatures and responses to immune checkpoint inhibitor (ICI) treatment, in accordance with an embodiment.

FIG. 3 is a graph of immunogenic signatures and traditional biomarkers, in accordance with an embodiment.

FIG. 4 is a diagram showing the ability of TIS and cell proliferation to predict response of a tumor to ICI, in accordance with an embodiment.

FIG. 5 is a graph of tumor response when TIS is used in conjunction with TMB and PD-L1 IHC, in accordance with an embodiment.

FIG. 6 is a diagram of tumor response when TIS is used in conjunction with TMB and PD-L1 IHC, in accordance with an embodiment.

FIG. 7 is a diagram showing an integrative hypothesis for utility of TIS and cell proliferation for treatment selection, in accordance with an embodiment.

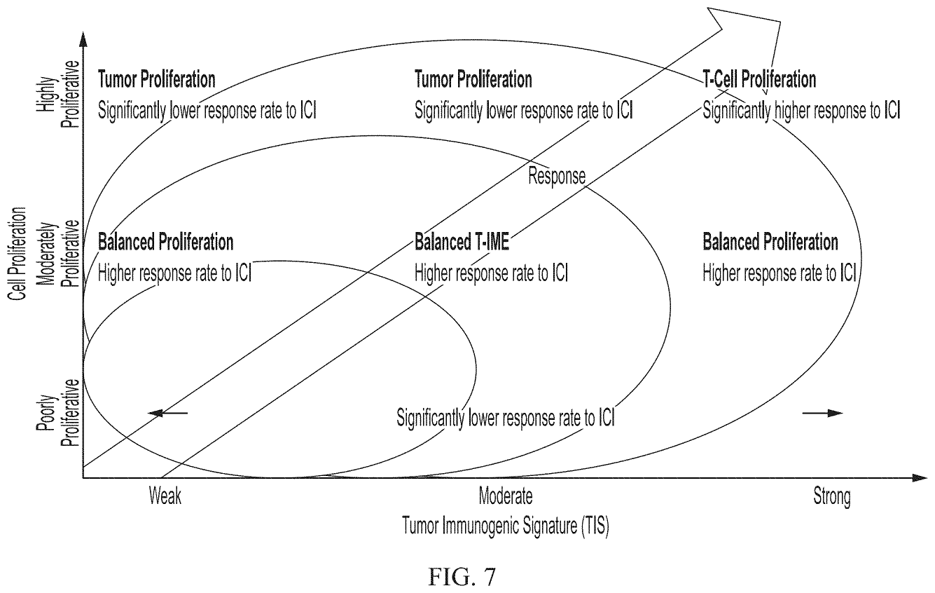

FIG. 8 is a diagram showing a gene expression rank calculation workflow, in accordance with an embodiment.

FIG. 9 is a diagram showing a tumor immunogenic signature discovery workflow, in accordance with an embodiment.

### DETAILED DESCRIPTION OF EMBODIMENTS

The present disclosure describes various embodiments of a system and method configured to identify a tumor as strongly immunogenic, moderately immunogenic, or weakly immunogenic. Applicant has recognized and appreciated that it would be beneficial to provide a method and system to characterize the response of a tumor to immunotherapy. The method utilizes tissue obtained from a tumor, which is used to generate an immune gene expression dataset comprising gene expression data for a plurality of immune genes. An immunogenic signature score is generated from the immune gene expression dataset, and the tumor is categorized as strongly immunogenic, moderately immunogenic, or weakly immunogenic based on the immunogenic signature score. The response of the tumor to immunotherapy can then be predicted based on the identification of the tumor as strongly immunogenic, moderately immunogenic, or weakly immunogenic. The response of the tumor to immunotherapy can also be based on one or more of a cell proliferation score, a PD-L 1 expression profile, and/or a tumor mutational burden (TMB) profile. Based on the predicted response of the tumor to immunotherapy, a clinician can determine a course of treatment for the tumor.

According to an embodiment, understanding immune tumor microenvironments (TMEs) can be crucial to the success of cancer immunotherapy. Reliance of immunotherapies on a robust host immune response necessitates clinical grade measurements of these immune TMEs for tumors. Accordingly, the methods described or otherwise envisioned herein provide a stable pan-cancer immunogenic profile, called an immunogenic score based on an obtained tumor immunogenic signature (TIS), is derived from RNA-sequencing expression data. The TIS is a comprehensive and informative measurement of immune TME that effectively describes host immune response to ICIs in NSCLC, melanoma, and RCC. The TIS is also applicable to PD-L1 and TMB-categorized tumors, and TIS combined with cell proliferation classification provides greater context of both immune and neoplastic influences on the tumor microenvironment. Further, TIS is able to discriminate subpopulations of responders to ICI that were negative for traditional biomarkers for response to ICI.

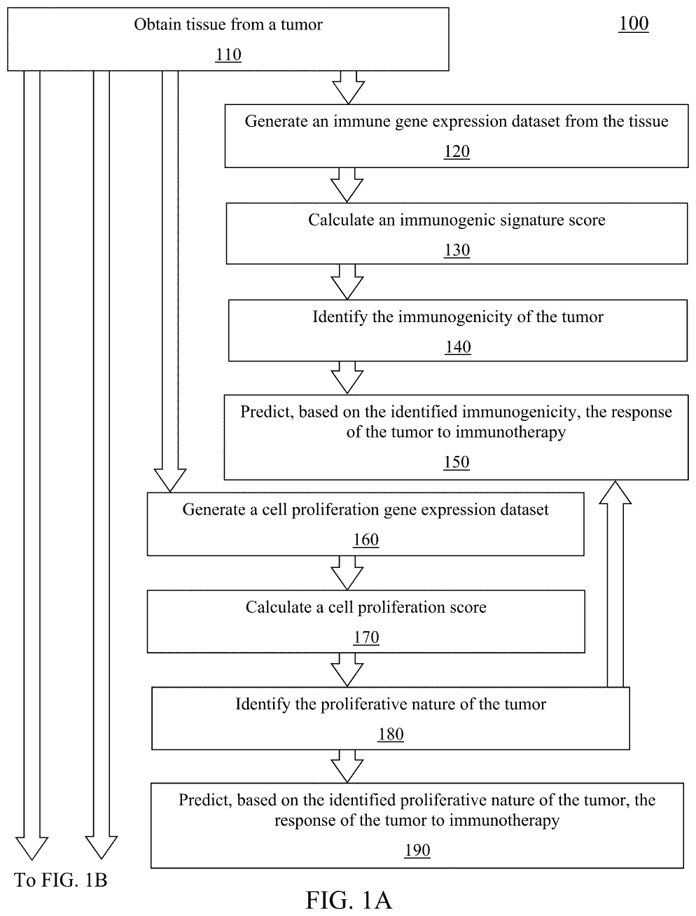

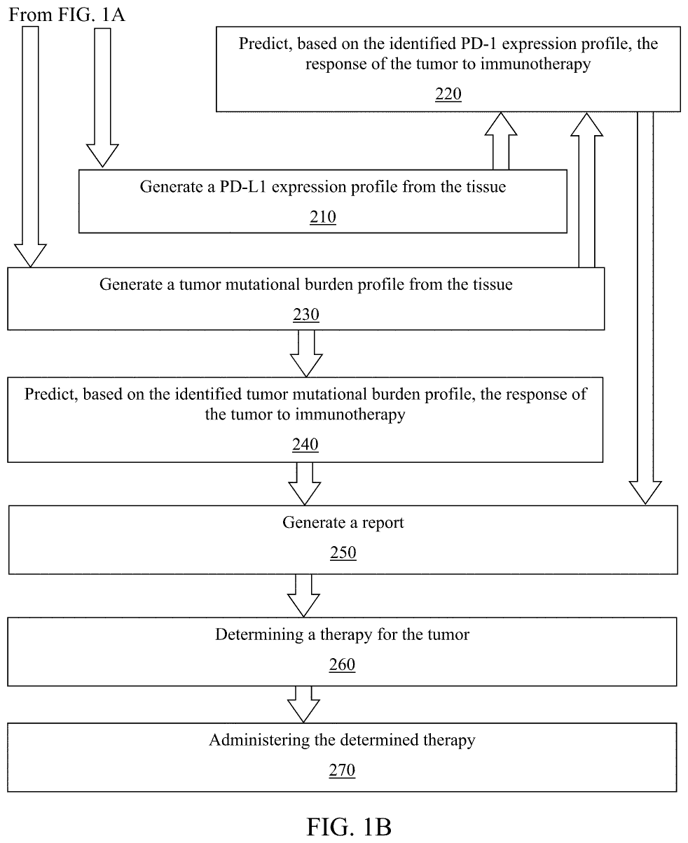

Referring to FIG. 1A, in one embodiment, is a flowchart of a method 100 for characterizing the response of a tumor to immunotherapy using a tumor analysis system. The tumor analysis system may be any of the systems described or otherwise envisioned herein.

At step 110 of the method, a tissue sample is obtained from a tumor or from a target which may potentially comprise a tumor. The tissue sample can be obtained using any method for obtaining tissue. The amount of tissue obtained may be dependent upon the intended use of the tissue, including but not limited to the uses described or otherwise envisioned herein. According to an embodiment, the tissue is obtained from a human, mammal, or other animal. The tissue may be utilized immediately for analysis, or may be stored for future use.

At step 120 of the method, an immune gene expression dataset is generated from the obtained tissue. The tissue may be processed using any method for processing tissue that yields a usable tissue or dataset for the tumor analysis system described or otherwise envisioned herein. According to an embodiment, the gene expression data is generated by RNA sequencing, although other methods are possible. According to an embodiment, the immune gene expression dataset comprises all gene expression data obtainable from the tissue. According to another embodiment, the immune gene expression dataset comprises only a subset of all gene expression data obtainable from the tissue, and may comprise only a specific analyzed subset of all immune or immune-related genes expressed or found within the tissue. For example, according to one embodiment, the immune gene expression dataset comprises expression data for the plurality of immune genes listed in Table 4, below. According to another embodiment, the immune gene expression dataset comprises expression data for only the 161 genes of Table 4. According to another embodiment, the immune gene expression dataset comprises expression data for a subset of the 161 genes of Table 4. According to another embodiment, the immune gene expression dataset comprises expression data for only a subset of the 161 genes of Table 4.

At step 130 of the method, the tumor analysis system generates an immunogenic score for the obtained tissue, and thus for the tumor, from the immune gene expression dataset. According to an embodiment, the immunogenic signature score comprises a mean expression rank for the gene expression data for the plurality of immune genes, although there are other methods for generating an immunogenic signature score from the immune gene expression dataset.

At step 140 of the method, the tumor analysis system uses the immunogenic score to identify the immunogenicity of the tumor. According to an embodiment, the tissue is identified as strongly immunogenic, moderately immunogenic, or weakly immunogenic based on the data from the immune gene expression dataset, although other categories are possible. According to an embodiment, clinically meaningful cutoffs for the immunogenic score can be generated by analyzing the average and standard deviation of the mean expression rank for the gene expression data for the plurality of immune genes, and the cutoffs for strongly immunogenicity (IS=62) can be derived as [Median IS]_{Borderline}+2×[Std. Dev. IS]_{Borderline}, and similarly, for weak immunogenicity (IS=43) was derived as [Median IS]_{Noninflamed}+2×[Std. Dev. IS]_{Noninflamed}, where IS=immunogenicity score. Any IS score between 62 and 43 can be classified as moderate immunogenicity. However, this is just an example and other cutoffs or other predetermined thresholds can be utilized.

At step 150 of the method, the tumor analysis system predicts the response of the tumor to immunotherapy based on the identified immunogenicity of the tumor. According to an embodiment, tissue/tumors identified as strongly immunogenic demonstrate an improved response rate to immune checkpoint inhibitors (ICIs), and thus tissue/tumor identified as strongly immunogenic is predicted to respond more favorably to immunotherapy. For example, a more favorable response may comprise a response to immunotherapy that is better than the response of tissue identified as anything other than strongly immunogenic. According to an embodiment, tissue/tumors identified as weakly immunogenic demonstrate a poor response rate to immune checkpoint inhibitors (ICIs), and thus tissue/tumor identified as weakly immunogenic is predicted to respond poorly or less favorably to immunotherapy. For example, a poor or less favorable response to immunotherapy may comprise a response to immunotherapy that is worse than the response of tissue identified as anything other than weakly immunogenic. According to an embodiment, tissue/tumors identified as moderately immunogenic demonstrate a response to immunotherapy that is better than tissue/tumors identified as weakly immunogenic but not as good a response as tissue/tumors identified as strongly immunogenic. Thus, for example, tissue/tumors identified as moderately immunogenic may be any tissue/tumor that is neither weakly nor strongly immunogenic.

At optional step 160 of the method, the tumor analysis system generates a cell proliferation gene expression dataset from the obtained tissue. The tissue may be processed using any method for processing tissue that yields a usable tissue or dataset for the tumor analysis system described or otherwise envisioned herein. According to an embodiment, the gene expression data is generated by RNA sequencing, although other methods are possible. According to an embodiment, the cell proliferation dataset comprises all gene expression data obtainable from the tissue. According to another embodiment, the cell proliferation dataset comprises only a subset of all gene expression data obtainable from the tissue, and may comprise only a specific analyzed subset of all cell proliferation genes expressed or found within the tissue. For example, according to one embodiment, the cell proliferation dataset comprises one or more of the following genes: BUB1, CCNB2, CDK1, CDKN3, FOXM1, KIAA0101, MAD2L1, MELK, MKI67, and/or TOP2A.

At optional step 170 of the method, the tumor analysis system generates a cell proliferation score for the obtained tissue, and thus for the tumor, from the cell proliferation gene expression dataset. The cell proliferation score may be generated using any method for analyzing the cell proliferation gene expression dataset. According to one embodiment, the cell proliferation score is calculated as the average gene expression rank of the genes utilized from the cell proliferation gene expression dataset (such as, for example, BUB1,CCNB2, CDK1, CDKN3, FOXM1, KIAA0101, MAD2L1, MELK, MKI67, and TOP2A). The cell proliferation score can be a number between 0-100.

At optional step 180 of the method, the tumor analysis system uses the cell proliferation score to identify the cell proliferative nature, or cell proliferative class, of the tissue and thus of the tumor. According to an embodiment, the tissue is identified as highly proliferative, moderately proliferative, or poorly proliferative based on the data from the cell proliferation score, although other categories are possible. According to one embodiment, tissue is identified as highly proliferative if the cell proliferation score is greater than or equal to 66, identified as moderately proliferative if the cell proliferation score is less than 66 and greater than or equal to 33, and identified as poorly proliferative if the cell proliferative score is less than 33. These as only example thresholds, and other thresholds may be utilized.

At optional step 190 of the method, the tumor analysis system predicts the response of the tumor to immunotherapy based on the identified cell proliferative class of the tumor. According to an embodiment, tissue/tumor identified as highly proliferative demonstrates an improved response rate to immune checkpoint inhibitors (ICIs), and thus tissue/tumor identified as highly proliferative is predicted to respond more favorably to immunotherapy. According to an embodiment, tissue tissue/tumor identified as poorly proliferative demonstrates a poor response to ICI therapy, and thus tissue/tumor identified as poorly proliferative is predicted to respond less favorably to immunotherapy. According to an embodiment, tissue/tumors identified as moderately proliferative demonstrate a response to immunotherapy that is better than tissue/tumors identified as weakly proliferative but not as good a response as tissue/tumors identified as strongly proliferative. Thus, for example, tissue/tumors identified as moderately proliferative may be any tissue/tumor that is neither weakly nor strongly proliferative.

According to an embodiment, at step 190 of the method, the tumor analysis system combines the result of step 180 of the method—identifying the proliferative nature of the tissue—with step 140 of the method—identifying the immunogenicity of the tissue—generate an improved predicted response of the tissue/tumor to immunotherapy. According to an embodiment, tumors that are identified as strongly immunogenic and highly proliferative have a significantly better predicted response to ICI therapy than tumors that are identified as weakly immunogenic and poorly proliferative. Further, tumors that are identified as strongly immunogenic and highly or moderately proliferative have a significantly better predicted response to ICI therapy than tumors that are identified as weakly immunogenic and highly proliferative.

Turning to the continuation of method 100 in FIG. 1B, at optional step 210 of the method, the tumor analysis system generates a PD-L1 expression profile from the obtained tissue. The tissue may be processed using any method for processing tissue that yields a usable tissue or dataset for the tumor analysis system described or otherwise envisioned herein. According to an embodiment, the gene expression data is generated by immunohistochemistry, although other methods are possible. According to an embodiment, the PD-L1 expression profile comprises a qualitative classification of the tissue as being PD-L1 positive or PD-L1 negative based on expression of PD-L1 in the tissue. According to one embodiment, a TPS≥1% is considered a positive expression (PD-L1+), and a PD-L1 TPS of <1% is considered a negative expression (PD-L1−). According to an embodiment, the gene expression data is quantitatively measured by RNA-sequencing, although other methods are possible. According to an embodiment, the PD-L1 expression profile comprises a classification of tissue as being PD-L1 percentile rank high or PD-L1 percentile rank low to moderate based on gene expression of PD-L1 in the tissue. According to one embodiment, percentile rank at or exceeding 75 is considered a PD-L1 positive expression (PD-L1+), and a PD-L1 percentile rank below 75 is considered a PD-L1 negative expression (PD-L1−).

At optional step 220 of the method, the tumor analysis system predicts the response of the tumor to immunotherapy based on the identified PD-L1 expression profile of the tumor. According to an embodiment, the tumor analysis system combines the result of the PD-L1 expression profile with the identified immunogenicity of the tissue to generate an improved predicted response of the tissue/tumor to immunotherapy. For example, tumors identified as PD-L1+ and strongly immunogenic have a significantly better predicted response to ICI therapy than tumors that are identified as moderately immunogenic or weakly immunogenic, and better than tumors identified as strongly immunogenic and PD-L1−.

At optional step 230 of the method, the tumor analysis system generates a tumor mutational burden (TMB) profile, wherein the TMB profile comprises mutational burden information about a plurality of genes generated from DNA sequencing data. The tissue may be processed using any method for processing tissue that yields a usable tissue or dataset for the tumor analysis system described or otherwise envisioned herein. According to an embodiment, the gene expression data is generated by DNA sequencing, although other methods are possible.

At optional step 240 of the method, the tumor analysis system predicts the response of the tumor to immunotherapy based on the identified TMB profile of the tumor. According to an embodiment, the tumor analysis system combines the result of the TMB profile with the identified immunogenicity of the tissue to generate an improved predicted response of the tissue/tumor to immunotherapy. For example, tumors identified as strongly immunogenic with a high TMB profile have a significantly better predicted response to ICI therapy than tumors that are identified as moderately immunogenic or weakly immunogenic, and better than tumors identified as strongly immunogenic with a low TMB profile.

According to an embodiment, the immunogenicity can be combined with both the TMB profile and the PD-L1 expression profile to predict response of a tumor to immunotherapy. For example, a tumor identified as strongly immunogenic with a high TMB and PD-L1+profile is more responsive to immunotherapy than a tumor identified as weakly immunogenic with a low TMB and PD-L1− profile.

At optional step 250 of the method, a report is generated by the tumor analysis system. According to an embodiment, the report comprises one or more of the following: (i) information about the patient, tumor, and/or tissue; (ii) information about the immune gene expression dataset; (iii) information about the immunogenic score; (iv) information about the identified immunogenicity of the tumor; (v) information about the cell proliferation gene expression dataset; (vi) information about the cell proliferation score; (vii) information about the cell proliferative class; (viii) information about the PD-L1 expression profile; (ix) information about the TMB profile; (x) a predicted response of the tissue/tumor to immunotherapy, where the predicted response is based on one or more of the identified immunogenicity of the tumor, the identified cell proliferative class, the PD-Le expression profile, and the TMB profile.

At optional step 260 of the method, a physician or other clinician utilizes the information about the predicted response of the tumor to immunotherapy, provided by the tumor analysis system, to determine or influence a course of action for treatment of the tumor. For example, the analysis provided by the tumor analysis system may indicate that the tumor is predicted to be highly responsive to immunotherapy, and the physician may thus determine a course of treatment that involves immunotherapy. As another example, the analysis provide by the tumor analysis system may indicate that the tumor is predicted to be weakly responsive or unresponsive to immunotherapy, and thus the physician may determine a course of treatment that involves something other than immunotherapy, or a treatment in addition to immunotherapy.

Accordingly, at step 270 of the method, the physician or other clinician administers the determined therapy. For example, the physician or other clinician may administer immunotherapy specific to the cancer type when the analysis by the tumor analysis system identifies the tumor as strongly immunogenic and/or moderately immunogenic. The determined therapy may be any immunotherapy suitable for the analyzed cancer type. For example, the determined immunotherapy may comprise a checkpoint inhibitor, antibody treatment, T-cell therapy, cancer vaccine, oncolytic virus, and/or any other cancer immunotherapy.

### EXAMPLE

Provided below is an example embodiment of the methods described or otherwise envisioned herein. It should be understood that the example application of the method described below does not limit the scope of the disclosure.

### Results

Although described in greater detail below, the results of the analysis described in this example are summarized briefly here. Unsupervised clustering of 1323 clinical RNA-seq profiles yielded three immunogenic clusters, namely, inflamed (n=439/1323; 33.18%), borderline (n=467/1323; 35.30%) and non-inflamed (n=417/1323; 31.52%). A 161 gene signature was over-represented by T cell and B cell activation pathways along with IFNg, chemokine, cytokine, and interleukin pathways. Mean expression of these 161 genes constituting the immunogenic signature produced an immunogenic score that led to three distinct groups of strong (n=384/1323; 29.02%), moderate (n=354/1323; 26.76%) and weak (n=585/1323; 44.22%) immunogenicity. Strongly inflamed tumors were over-represented by PD-L1⁺ tumors (240/384), whereas weakly inflamed tumors were significantly under-represented by PD-L1⁻ tumors (369/585; p=1.023e-14). Strongly inflamed tumors presented with improved response rate of 37% (30/81) to immune checkpoint inhibitors (ICIs) in pan-cancer retrospective cohort compared to weakly inflamed tumors (21/92; p=0.06031); with highest response rate advantage occurring in NSCLC (ORR=36.6%; 16/44; P=0.051) and not in melanoma (ORR=52.94%; 9/17; p=0.2784) or RCC (ORR=25.0%; 5/20; p=0.8176). Similar results were observed for overall survival in retrospective cohort, where, strongly inflamed tumors trended towards improved survival (median=25 months; p=0.19) in pan-cancer cohort. However, in tumor specific analyses, significantly higher survival was only observed in NSCLC for strongly inflamed tumors (median=16 months; p=0.0012). Integrating TIS groups with cell proliferation classes showed highly proliferative and inflamed tumors have significantly higher objective response to ICIs than poorly proliferative and non-inflamed tumors [14.28%; p=0.0006].

### Methods—Patients and Clinical Data

The study involved two separate cohorts, namely, a discovery cohort of clinical tumors used for development of an immunogenic signature and a retrospective cohort for which information about the response of the tumor to ICI therapy was available. For the discovery cohort, a total of 1323 patients were included in the study, based on the following criteria: (1) availability of high-quality gene expression data from samples clinically tested by a CLIA approved targeted RNA-seq assay; (2) samples that pass clinically approved tissue, nucleic acid, and sequencing QC metrics; (3) samples that have less than 50% necrosis and at least 5% tumor purity; and (4) availability of other primary immune biomarkers such as PD-L1 IHC (TPS %) and TMB (Mut/Mb). TABLE 1 summarizes the baseline clinical characteristics of these patients.

TABLE 1

| | Lung Cancer | | Melanoma | | | | | | | |
|---|---|---|---|---|---|---|---|---|---|---|
| | Patients | | | | Pre-ipi | | Post-ipi | | RCC | |
| | Patients | | All Cases | | approval | | approval | | ICI Treated | |
| | (n = 110) | | (n = 78) | | (n = 4) | | (n = 74) | | (n = 54) | |
| Age at initial | | | | | | | | | | |
| diagnosis (years) | | | | | | | | | | |
| <30 | 1 | (0.9%) | | | | | | | | |
| 30-39 | | | 7 | (9.0%) | 1 | (25.0%) | 6 | (8.1%) | 1 | (1.9%) |
| 40-49 | 3 | (2.7%) | 14 | (17.9%) | 1 | (25.0%) | 13 | (17.6%) | 6 | (11.1%) |
| 50-59 | 26 | (23.6%) | 13 | (16.7%) | 1 | (25.0%) | 12 | (16.2%) | 21 | (38.9%) |
| 60-69 | 41 | (37.3%) | 19 | (24.4%) | 1 | (25.0%) | 18 | (24.3%) | 16 | (29.6%) |
| 70-79 | 30 | (27.3%) | 18 | (23.1%) | | | 18 | (24.3%) | 10 | (18.5%) |
| ≥80 | 9 | (8.2%) | 7 | (9.0%) | | | 7 | (9.5%) | | |
| Mean | 65.4 | | 60.6 | | 48 | | 61.3 | | 59.5 | |
| Year of diagnosis | 2007-2017 | | 1990-2016 | | 2004-2009 | | 1990-2016 | | 1981-2016 | |
| (Range) | | | | | | | | | | |
| Sex | | | | | | | | | | |
| Female | 58 | (52.7%) | 26 | (33.3%) | 2 | (50.0%) | 24 | (32.4%) | 14 | (25.9%) |
| Male | 52 | (47.3%) | 52 | (66.7%) | 2 | (50.0%) | 50 | (67.6%) | 40 | (74.1%) |
| Race | | | | | | | | | | |
| White | 91 | (82.7%) | 78 | (100.0%) | 4 | (100.0%) | 74 | (100.0%) | 41 | (5.9%) |
| Other | 14 | (12.7%) | | | | | | | 7 | (13.0%) |
| Unknown | 5 | (4.5%) | | | | | | | 6 | (11.1%) |
| Vital status at last | | | | | | | | | | |
| follow up | | | | | | | | | | |
| Alive | 55.00 | (50.0%) | 46.00 | (59.0%) | 2.00 | (50.0%) | 44.00 | (59.5%) | 31.0 | (57.4%) |
| Dead | 55.00 | (50.0%) | 32.00 | (41.0%) | 2.00 | (50.0%) | 30.00 | (40.5%) | 23.00 | (42.6%) |
| Checkpoint | | | | | | | | | | |
| inhibitor | | | | | | | | | | |
| atezolizumab | 2 | (1.8%) | | | | | | | | |
| ipilimumab | | | 35 | (44.9%) | 3 | (75.0%) | 32 | (43.2%) | | |
| ipilimumab + | 2 | (1.8%) | 10 | (12.8%) | 1 | (25.0%) | 9 | (12.2%) | | |
| nivolumab | | | | | | | | | | |
| nivolumab | 71 | (64.5%) | 2 | (2.6%) | | | 2 | (2.7%) | 54 | (100.0%) |
| pembrolizumab | 35 | (31.8%) | 31 | (39.7%) | | | 31 | (41.9%) | | |
| Months of follow up | | | | | | | | | | |
| <1 | 48 | (43.6%) | | | | | | | 21 | (38.9%) |
| 3 | 6 | (5.5%) | 1 | (1.3%) | | | 1 | (1.4%) | 1 | (1.9%) |
| 6 | 17 | (15.5%) | 12 | (15.4%) | | | 12 | (16.2%) | 5 | (9.3%) |
| 10 | 22 | (20.0%) | 15 | (19.2%) | | | 15 | (20.3%) | 14 | (25.9%) |
| >10 | 17 | (15.5%) | 50 | (64.1%) | 4 | (100.0%) | 46 | (62.2%) | 13 | (24.1%) |

| | | | | | |
|---|---|---|---|---|---|
| Median | 8 | 12.5 | 63 | 12 | 10 |

The retrospective cohort of 242 cases were from patients treated with ICIs including non-small cell lung cancer cases (n=110), melanoma (n=78) and renal cell carcinoma cases (n=54). Inclusion criteria comprised of treatment by an FDA approved ICI agent as of November 2017 and had follow up and survival from first ICI dose (n=242). Additionally, evaluable response based on RECIST v1.1 was available on all 242 cases. RECIST responses of complete response (CR) and partial response (PR) were classified as responders, whereas, stable disease (SD) or progressive disease (PD) were classified as non-responders. Duration of response was not available for all patients and not included for final analysis.

### Methods—Quality Assessment of Clinical FFPE Tissue Specimens

Tissue sections from FFPE blocks were cut at 5 μm onto positively charged slides. One cut section from each tissue sample was stained with H&E and assessed by a board-certified anatomical pathologist for adequacy of tumor representation, the quality of tissue preservation, evidence of necrosis, or issues with fixation or handling were present. Specimens containing <5% tumor tissue and >50% necrosis were excluded from analysis. In general, tissue from 3-5 unstained slide sections, with or without tumor macrodissection, was required to achieve the assay requirements for RNA (10 ng) and DNA (20 ng) input.

### Methods—Immunohistochemical Studies

The expression of PD-L1 on the surface of cancer cells was assessed in all cases regardless of tumor type by means of the Dako PD-L1 IHC 22C3 pharmDx (Agilent, Santa Clara, Calif.). PD-L1 levels were scored by a board-certified anatomic pathologist as per published guidelines, with a TPS >1% considered as positive result (PD-L1+). PD-L1 TPS <1% was considered negative (PD-L1−).

Tissue sections were also examined for CD8 T-cell infiltration using anti-CD8 antibodies (C8/144B; Agilent, Santa Clara, Calif.) and classified into non-infiltrating, infiltrating, or excluded CD8 infiltration groups. Cases where a sparse number of CD8+T-cells infiltrated clusters of neoplastic cells with less than 5% of the tumor showing an infiltrating pattern were designated non-infiltrating, while those showing frequent infiltration of neoplastic cell clusters in an overlapping fashion, at least focally, in more than 5% of the tumor were designated infiltrating. Cases where more than 95% of CD8+T-cells were restricted to the tumor periphery or interstitial stromal areas and did not actively invade clusters of neoplastic cells were designated as excluded.

### Methods—Nucleic Acid Isolation, Gene Expression, and TMB

DNA and RNA were co-extracted from each sample and processed for gene expression by RNA-seq and TMB by DNA-seq. Nucleic acids were quantitated by Qubit fluorometer (Thermo Fisher Scientific) using ribogreen staining for RNA and picogreen staining for DNA. Gene expression were evaluated by RNA sequencing of 395 transcripts on samples that met validated quality control (QC) thresholds. TMB was measured by DNA sequencing of the full coding region of 409 cancer related genes as non-synonymous mutations per megabase (Mut/Mb) of sequenced DNA on samples with >30% tumor nuclei (see Table 2). However, the list of genes utilized for TMB may be different than the list in Table 2, and may be more or fewer than the genes listed in Table 2. RNA and DNA libraries were sequenced to appropriate depth on the Ion Torrent SSXL sequencer (Thermo Fisher Scientific).

TABLE 2
TMB gene list

SEP9
ABL1
ABL2
ACVR2A
ADAMTS20
AFF1
AFF3
AKAP9
AKT1
AKT2
AKT3
ALK
APC
AR
ARID1A
ARID2
ARNT
ASXL1
ATF1
ATM
ATR
ATRX
AURKA
AURKB
AURKC
AXL
BAI3
BAP1
BCL10
BCL11A
BCL11B
BCL2
BCL2L1
BCL2L2
BCL3
BCL6
BCL9
BCR
BIRC2
BIRC3
BIRC5
BLM
BLNK
BMPR1A
BRAF
BRD3
BTK
BUB1B
CARD11
CASC5
CBL
CCND1
CCND2
CCNE1
CD79A
CD79B
CDC73
CDH1
CDH11
CDH2
CDH20
CDH5
CDK12
CDK4
CDK6
CDK8
CDKN2A
CDKN2B
CDKN2C
CEBPA
CHEK1
CHEK2
CIC
CKS1B
CMPK1
COL1A1
CRBN
CREB1
CREBBP
CRKL
CRTC1
CSF1R
CSMD3
CTNNA1
CTNNB1
CYLD
CYP2C19
CYP2D6
DAXX
DCC
DDB2
DDIT3
DDR2
DEK
DICER1
DNMT3A
DPYD
DST
EGFR
EML4
EP300
EP400
EPHA3
EPHA7
EPHB1
EPHB4
EPHB6
ERBB2
ERBB3
ERBB4
ERCC1
ERCC2
ERCC3
ERCC4
ERCC5
ERG
ESR1
ETS1
ETV1
ETV4
EXT1
EXT2
EZH2
FAM123B
FANCA
FANCC
FANCD2
FANCF
FANCG
FANCJ
FAS
FBXW7
FGFR1
FGFR2
FGFR3
FGFR4
FH
FLCN
FLI1
FLT1
FLT3
FLT4
FN1
FOXL2
FOXO1
FOXO3
FOXP1
FOXP4
FZR1
G6PD
GATA1
GATA2
GATA3
GDNF
GNA11
GNAQ
GNAS
GPR124
GRM8
GUCY1A2
HCAR1
HIF1A
HLF
HNF1A
HOOK3
HRAS
HSP90AA1
HSP90AB1
ICK
IDH1
IDH2
IGF1R
IGF2
IGF2R
IKBKB
IKBKE
IKZF1
IL2
IL21R
IL6ST
IL7R
ING4
IRF4
IRS2
ITGA10
ITGA9
ITGB2
ITGB3
JAK1
JAK2
JAK3
JUN
KAT6A
KAT6B
KDM5C
KDM6A
KDR
KEAP1
KIT
KLF6
KRAS
LAMP1
LCK
LIFR
LPHN3
LPP
LRP1B
LTF
LTK
MAF
MAFB
MAGEA1
MAGI1
MALT1
MAML2
MAP2K1
MAP2K2
MAP2K4
MAP3K7
MAPK1
MAPK8
MARK1
MARK4
MBD1
MCL1
MDM2
MDM4
MEN1
MET
MITF
MLH1
MLL
MLL2
MLL3
MLLT10
MMP2
MN1
MPL
MRE11A
MSH2
MSH6
MTOR
MTR
MTRR
MUC1
MUTYH
MYB
MYC
MYCL1
MYCN
MYD88
MYH11
MYH9
NBN
NCOA1
NCOA2
NCOA4
NF1
NF2
NFE2L2
NFKB1
NFKB2
NIN
NKX2-1
NLRP1
NOTCH1
NOTCH2
NOTCH4
NPM1
NRAS
NSD1
NTRK1
NTRK3
NUMA1
NUP214
NUP98
PAK3
PALB2
PARP1
PAX3
PAX5
PAX7
PAX8
PBRM1
PBX1
PDE4DIP
PDGFB
PDGFRA
PDGFRB
PER1
PGAP3
PHOX2B
PIK3C2B
PIK3CA
PIK3CB
PIK3CD
PIK3CG
PIK3R1
PIK3R2
PIM1
PKHD1
PLAG1
PLCG1
PLEKHG5
PML
PMS1
PMS2
POT1
POU5F1
PPARG
PPP2R1A
PRDM1
PRKAR1A
PRKDC
PSIP1
PTCH1
PTEN
PTGS2
PTPN11
PTPRD
PTPRT
RAD50
RAF1
RALGDS
RARA
RB1
RECQL4
REL
RET
RHOH
RNASEL
RNF2
RNF213
ROS1
RPS6KA2
RRM1
RUNX1
RUNX1T1
SAMD9
SBDS
SDHA
SDHB
SDHC
SDHD
SETD2
SF3B1
SGK1
SH2D1A
SMAD2
SMAD4
SMARCA4
SMARCB1
SMO
SMUG1
SOCS1
SOX11
SOX2
SRC
SSX1
STK11
STK36
SUFU
SYK
SYNE1
TAF1
TAF1L
TALI
TBX22
TCF12
TCF3
TCF7L1
TCF7L2
TCL1A
TET1
TET2
TFE3
TGFBR2
TGM7
THBS1
TIMP3
TLR4
TLX1
TNFAIP3
TNFRSF14
TNK2
TOP1
TP53
TPR
TRIM24
TRIM33
TRIP11
TRRAP
TSC1
TSC2
TSHR
UBR5
UGT1A1
USP9X
VHL
WAS
WHSC1
WRN
WT1
XPA
XPC
XPO1
XRCC2
ZNF384
ZNF521

For example, in accordance with another embodiment, TMB can be measured by DNA sequencing of another set of genes, which may be, for example, cancer-related genes. Cancer-related genes may be any two or more genes identified as being involved or believed to be involved with cancer, including as a regulator of, inhibitor of, activator of, signal of, or otherwise involved in, cancer. For example, TMB can be measured by DNA analysis of all of the genes listed in the gene set of Table 3, or only some of the genes listed in the gene set of Table 3.

TABLE 3
TMB gene list

ABL1
ABL2
ACVR1
ACVR1B
AKT1
AKT2
AKT3
ALK
ALOX12B
ANKRD11
ANKRD26
APC
AR
ARAF
ARFRP1
ARID1A
ARID1B
ARID2
ARID5B
ASXL1
ASXL2
ATM
ATR
ATRX
AURKA
AURKB
AXIN1
AXIN2
AXL
B2M
BAP1
BARD1
BBC3
BCL10
BCL2
BCL2L1
BCL2L11
BCL2L2
BCL6
BCOR
BCORL1
BCR
BIRC3
BLM
BMPR1A
BRAF
BRCA1
BRCA2
BRD4
BRIP1
BTG1
BTK
C11orf30
CALR
CARD11
CASP8
CBFB
CBL
CCND1
CCND2
CCND3
CCNE1
CD274
CD276
CD74
CD79A
CD79B
CDC73
CDH1
CDK12
CDK4
CDK6
CDK8
CDKN1A
CDKN1B
CDKN2A
CDKN2B
CDKN2C
CEBPA
CENPA
CHD2
CHD4
CHEK1
CHEK2
CIC
CREBBP
CRKL
CRLF2
CSF1R
CSF3R
CSNK1A1
CTCF
CTLA4
CTNNA1
CTNNB1
CUL3
CUX1
CXCR4
CYLD
DAXX
DCUN1D1
DDR2
DDX41
DHX15
DICER1
DIS3
DNAJB1
DNMT1
DNMT3A
DNMT3B
DOT1L
E2F3
EED
EGFL7
EGFR
EIF1AX
EIF4A2
EIF4E
EML4
EP300
EPCAM
EPHA3
EPHA5
EPHA7
EPHB1
ERBB2
ERBB3
ERBB4
ERCC1
ERCC2
ERCC3
ERCC4
ERCC5
ERG
ERRFI1
ESR1
ETS1
ETV1
ETV4
ETV5
ETV6
EWSR1
EZH2
FAM123B
FAM175A
FAM46C
FANCA
FANCC
FANCD2
FANCE
FANCF
FANCG
FANCI
FANCL
FAS
FAT1
FBXW7
FGF1
FGF10
FGF14
FGF19
FGF2
FGF23
FGF3
FGF4
FGF5
FGF6
FGF7
FGF8
FGF9
FGFR1
FGFR2
FGFR3
FGFR4
FH
FLCN
FLI1
FLT1
FLT3
FLT4
FOXA1
FOXL2
FOXO1
FOXP1
FRS2
FUBP1
FYN
GABRA6
GATA1
GATA2
GATA3
GATA4
GATA6
GEN1
GID4
GLI1
GNA11
GNA13
GNAQ
GNAS
GPR124
GPS2
GREM1
GRIN2A
GRM3
GSK3B
H3F3A
H3F3B
H3F3C
HGF
HIST1H1C
HIST1H2BD
HIST1H3A
HIST1H3B
HIST1H3C
HIST1H3D
HIST1H3E
HIST1H3F
HIST1H3G
HIST1H3H
HIST1H3I
HIST1H3J
HIST2H3A
HIST2H3C
HIST2H3D
HIST3H3
HLA-A
HLA-B
HLA-C
HNF1A
HNRNPK
HOXB13
HRAS
HSD3B1
HSP90AA1
ICOSLG
ID3
IDH1
IDH2
IFNGR1
IGF1
IGF1R
IGF2
IKBKE
IKZF1
IL10
IL7R
INHA
INHBA
INPP4A
INPP4B
INSR
IRF2
IRF4
IRS1
IRS2
JAK1
JAK2
JAK3
JUN
KAT6A
KDM5A
KDM5C
KDM6A
KDR
KEAP1
KEL
KIF5B
KIT
KLF4
KLHL6
KMT2B
KMT2C
KMT2D
KRAS
LAMP1
LATS1
LATS2
LMO1
LRP1B
LYN
LZTR1
MAGI2
MALT1
MAP2K1
MAP2K2
MAP2K4
MAP3K1
MAP3K13
MAP3K14
MAP3K4
MAPK1
MAPK3
MAX
MCL1
MDC1
MDM2
MDM4
MED12
MEF2B
MEN1
MET
MGA
MITF
MLH1
MLL
MLLT3
MPL
MRE11A
MSH2
MSH3
MSH6
MST1
MST1R
MTOR
MUTYH
MYB
MYC
MYCL1
MYCN
MYD88
MYOD1
NAB2
NBN
NCOA3
NCOR1
NEGR1
NF1
NF2
NFE2L2
NFKBIA
NKX2-1
NKX3-1
NOTCH1
NOTCH2
NOTCH3
NOTCH4
NPM1
NRAS
NRG1
NSD1
NTRK1
NTRK2
NTRK3
NUP93
NUTM1
PAK1
PAK3
PAK7
PALB2
PARK2
PARP1
PAX3
PAX5
PAX7
PAX8
PBRM1
PDCD1
PDCD1LG2
PDGFRA
PDGFRB
PDK1
PDPK1
PGR
PHF6
PHOX2B
PIK3C2B
PIK3C2G
PIK3C3
PIK3CA
PIK3CB
PIK3CD
PIK3CG
PIK3R1
PIK3R2
PIK3R3
PIM1
PLCG2
PLK2
PMAIP1
PMS1
PMS2
PNRC1
POLD1
POLE
PPARG
PPM1D
PPP2R1A
PPP2R2A
PPP6C
PRDM1
PREX2
PRKAR1A
PRKCI
PRKDC
PRSS8
PTCH1
PTEN
PTPN11
PTPRD
PTPRS
PTPRT
QKI
RAB35
RAC1
RAD21
RAD50
RAD51
RAD51B
RAD51C
RAD51D
RAD52
RAD54L
RAF1
RANBP2
RARA
RASA1
RB1
RBM10
RECQL4
REL
RET
RFWD2
RHEB
RHOA
RICTOR
RIT1
RNF43
ROS1
RPS6KA4
RPS6KB1
RPS6KB2
RPTOR
RUNX1
RUNX1T1
RYBP
SDHA
SDHAF2
SDHB
SDHC
SDHD
SETBP1
SETD2
SF3B1
SH2B3
SH2D1A
SHQ1
SLIT2
SLX4
SMAD2
SMAD3
SMAD4
SMARCA4
SMARCB1
SMARCD1
SMC1A
SMC3
SMO
SNCAIP
SOCS1
SOX10
SOX17
SOX2
SOX9
SPEN
SPOP
SPTA1
SRC
SRSF2
STAG1
STAG2
STAT3
STAT4
STAT5A
STAT5B
STK11
STK40
SUFU
SUZ12
SYK
TAF1
TBX3
TCEB1
TCF3
TCF7L2
TERC
TERT
TET1
TET2
TFE3
TFRC
TGFBR1
TGFBR2
TMEM127
TMPRSS2
TNFAIP3
TNFRSF14
TOP1
TOP2A
TP53
TP63
TRAF2
TRAF7
TSC1
TSC2
TSHR
U2AF1
VEGFA
VHL
VTCN1
WISP3
WT1
XIAP
XPO1
XRCC2
YAP1
YES1
ZBTB2
ZBTB7A
ZFHX3
ZNF217
ZNF703
ZRSR2

### Methods—Data Analyses

Using the Torrent Suite plugin immuneResponseRNA (Thermo Fisher Scientific), RNA-seq absolute reads were generated for each transcript. In each case, absolute read counts from the NTC were used as the library preparation background which was subtracted from the absolute read counts of the same transcript in all other samples of the same batch. To facilitate the comparability of NGS measurements across runs for evaluation and interpretation, background-subtracted read counts were normalized into nRPM values by comparing each HK gene background-subtracted read against an already-determined HK RPM profile. This HK RPM profile was calculated as the average RPM of multiple GM12878 sample replicates across different validation sequencing runs, producing the following fold-change ration for each HK gene:

$$Ratio of HK = \frac{Background Subtracted Read Count of HK}{RPM Profile of HK}$$

After this, the median value of all HK ratios was used as the normalization ratio for each sample. Following from this, the nRPM of all genes (G) of a specific sample (S) were then calculated as:

$${nRPM}_{( S , G )} = \frac{Background Subtracted Read {Count}_{( S , G )}}{Normalization {Ratio}_{( S )}}$$

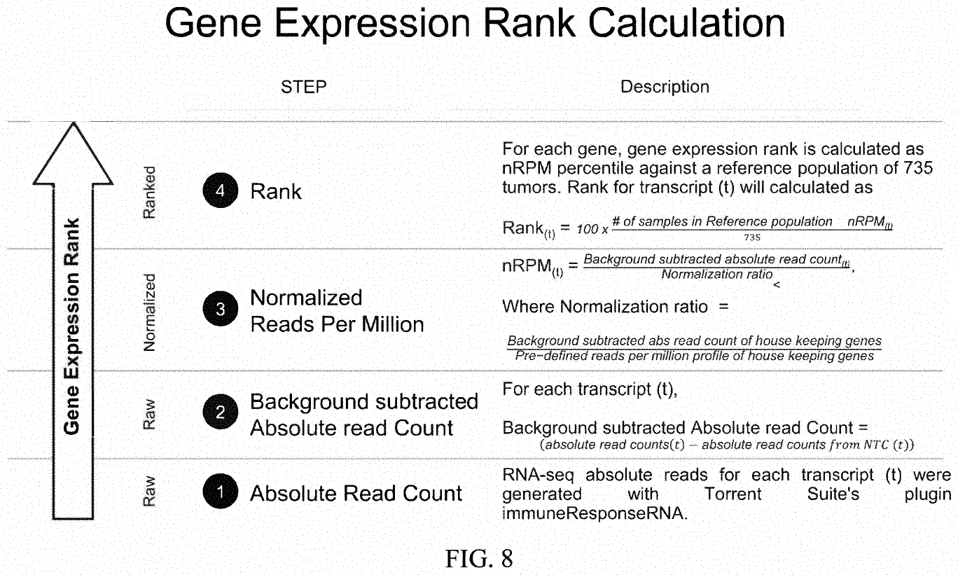

For each gene, nRPM expression values are converted to percentile rank of 0-100 when compared to a reference population of 735 solid tumors of 35 histologies. See, FIG. 8 for a gene expression rank calculation workflow.

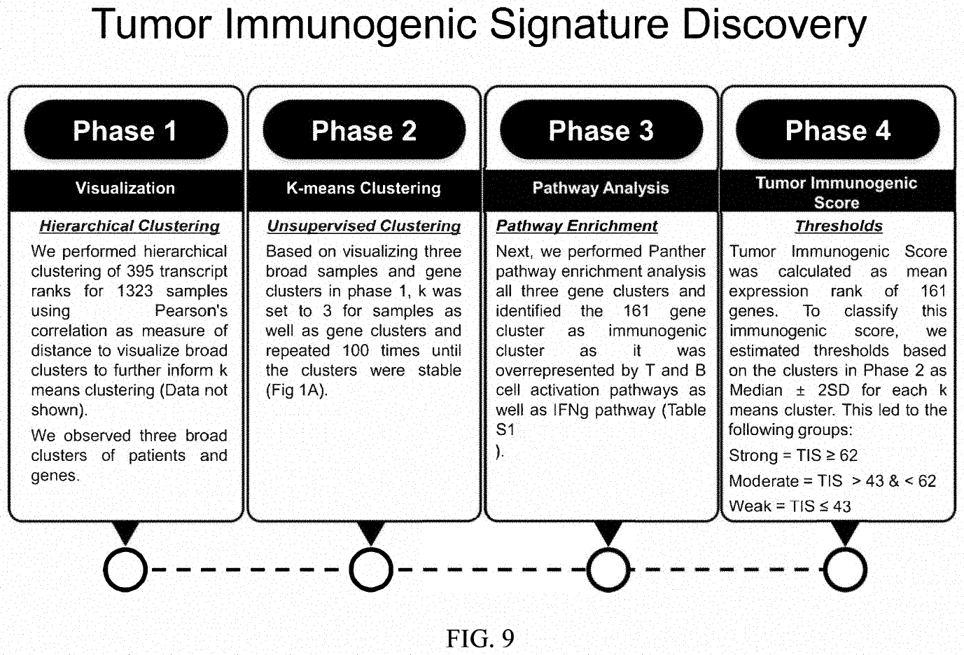

Initial visualization of the overall gene expression landscape of the discovery cohort was performed on the gene expression rank values using unsupervised hierarchical clustering with Pearson's correlation (R) used as a measure of distance. These results were then refined using k-means (k=3) clustering to generate three stable clusters of patients. Pathway enrichment analysis of these gene clusters distinguished them as cancer testis antigen genes, genes associated with the inflammation response, and other immune and neoplasm genes (see TABLES 5 and 6). The 161-gene cluster associated with the inflammation response was termed the immunogenic signature, as the expression of these genes closely followed the degree of inflammation presented by each of the three patient clusters. See FIG. 9 for a tumor immunogenic signature discovery workflow.

For each patient, the immunogenic score (IS) was calculated as mean expression rank of these 161 transcripts. To derive clinically meaningful cutoffs for immunogenic score, overall average and standard deviation of immunogenic score was calculated across the three patients cluster of inflamed, borderline, and non-inflamed tumors (see, Table 7). Cutoff for strong immunogenicity (IS=62) was derived as [Median IS]_{Borderline}+2×[Std. Dev. IS]_{Bordedine}, and similarly, for weak immunogenicity (IS=43) was derived as, [Median IS]_{Noninflamed}+2×[Std. Dev. IS]_{Noninflamed}, where IS=immunogenicity score. Any IS score between 62 and 43 was classified as moderate immunogenicity. For the retrospective cohort with clinical outcome and survival data, survival analyses were performed using a log-rank test on 5-year Kaplan-Meier survival curves. Comparison of ICI response rates was performed using Chi-square test with Yate's continuity correction to test for significant differences in ICI response for various biomarker groups. See FIG. 9

This resulted in three broad clusters of patients (data not shown) used to inform a second k-means (k=3, repeat=100) clustering step to better group genes and patients into stable clusters. Gene cluster number 2 contained 161 genes and closely represented the overall immunogenic landscape of the three-patient clusters (inflamed, borderline and non-inflamed) and therefore was designated as the “immunogenic signature.” The 161 genes in this immunogenic signature are identified in TABLE 4.

TABLE 4

ADORA2A
AIF1
B3GAT1
BATF
BTLA
C1QA
C1QB
CCL17
CCL21
CCL4
CCL5
CCR2
CCR4
CCR5
CCR6
CCR7
CD160
CD19
CD1C
CD1D
CD2
CD22
CD226
CD244
CD247
CD27
CD274
CD28
CD3
CD37
CD38
CD3D
CD3E
CD3G
CD40
CD40LG
CD48
CD52
CD53
CD6
CD69
CD70
CD79A
CD79B
CD8
CD80
CD83
CD8A
CD8B
CIITA
CORO1A
CRTAM
CSF2RB
CTLA4
CXCL10
CXCL11
CXCL13
CXCL9
CXCR3
CXCR5
CXCR6
CYBB
EBI3
EOMES
FASLG
FCGR2B
FOXP3
FYB
GATA3
GBP1
GNLY
GPR18
GRAP2
GZMA
GZMB
GZMH
GZMK
HAVCR2
HLA-A
HLA-C
HLA-DMA
HLA-DOA
HLA-DOB
HLA-DPA1
HLA-DPB1
HLA-DQA2
HLA-DQB2
HLA-DRA
HLA-E
HLA-F
ICOS
IDO1
IFNB1
IFNG
IKZF1
IKZF3
IL10RA
IL2RA
IL2RB
IL2RG
IL7
IL7R
IRF4
ISG20
ITGAL
ITGAM
ITGAX
ITGB7
ITK
JAML
JCHAIN
KLF2
KLRB1
KLRD1
KLRF1
KLRG1
KLRK1
LAG3
LCK
LILRB1
LILRB2
LY9
LYZ
M6PR
MPO
MS4A1
NCF1
NCR1
NCR3
NFATC1
NKG7
PDCD1
PIK3CD
POU2AF1
PRF1
PTPN6
PTPN7
PTPRC
PTPRCAP
SH2D1A
SH2D1B
SIT1
SLAMF7
SLAMF8
SRGN
STAT1
STAT4
STAT5A
TAGAP
TARP
TBX21
TCF7
TIGIT
TLR8
TLR9
TNFAIP8
TNFRSF17
TNFRSF4
TNFRSF9
TNFSF14
ZAP70

### Results—Tumor Immunogenic Signature (TIS)

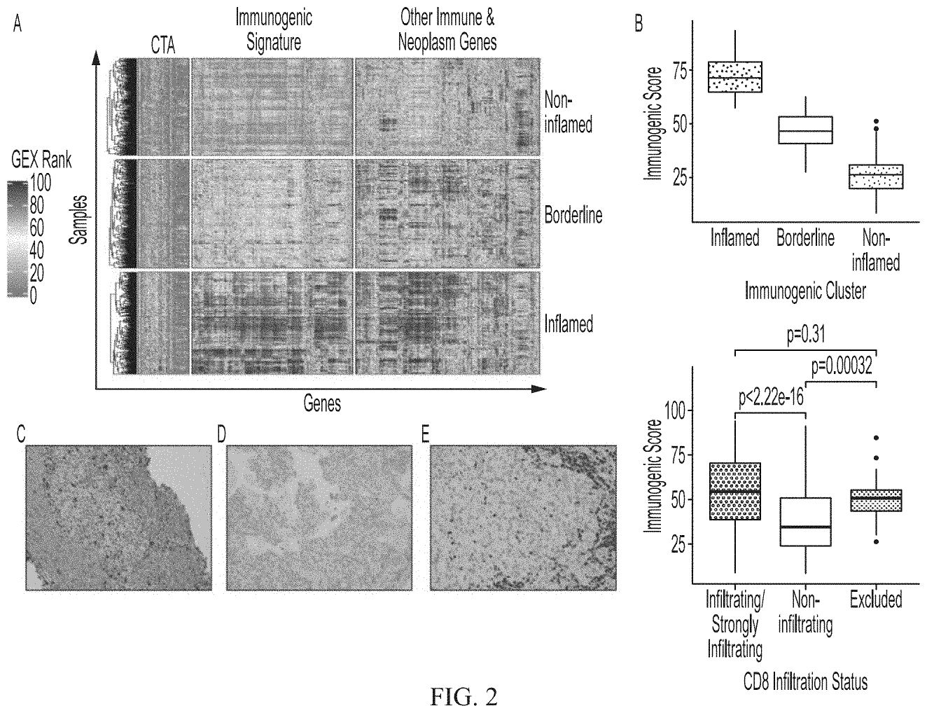

Unsupervised hierarchical clustering of all genes sequenced in the discovery cohort revealed three clusters of coexpressing genes. Refining these results using k-means (k=3) clustering generated three stable clusters of genes and three clusters of patients (inflamed, borderline, and noninflamed) shown in FIG. 2. Pathway analysis of these gene clusters distinguished them as cancer testis antigen genes, genes associated with the inflammation response, and other immune and neoplasm genes (see TABLES 5 and 6). The 161 genes associated with the inflammation response were termed the immunogenic signature, as the expression of these genes closely followed the degree of inflammation presented by each of the three patient clusters. The distributions of the immunogenic scores of all samples in each of sample cluster were used to establish boundaries between three immunogenic score groups (strong, moderate, and weak).

TABLE 5
Pathway analysis of genes in immunogenic signature cluster.

| | Homo | | | | | | |
|---|---|---|---|---|---|---|---|
| | sapiens - | Client Text | Client Text | | | | |
| PANTHER | REFLIST | Box Input | Box Input | | Fold | Raw | |
| Pathways | (20996) | (163) | (Expected) | Over/Under | Enrichment | P-value | FDR |
| JAK/STAT | 17 | 4 | 0.13 | + | 30.31 | 1.83E−05 | 5.01E−04 |
| signaling | | | | | | | |
| pathway | | | | | | | |
| (P00038) | | | | | | | |
| T cell | 95 | 17 | 0.74 | + | 23.05 | 1.45E−17 | 2.38E−15 |
| activation | | | | | | | |
| (P00053) | | | | | | | |
| B cell | 72 | 8 | 0.56 | + | 14.31 | 1.89E−07 | 7.75E−06 |
| activation | | | | | | | |
| (P00010) | | | | | | | |
| Interferon- | 29 | 3 | 0.23 | + | 13.33 | 1.89E−03 | 4.43E−02 |
| gamma | | | | | | | |
| signaling | | | | | | | |
| pathway | | | | | | | |
| (P00035) | | | | | | | |
| Inflammation | 260 | 21 | 2.02 | + | 10.4 | 4.93E−15 | 4.04E−13 |
| mediated by | | | | | | | |
| chemokine | | | | | | | |
| and cytokine | | | | | | | |
| signaling | | | | | | | |
| pathway | | | | | | | |
| (P00031) | | | | | | | |
| Interleukin | 89 | 7 | 0.69 | + | 10.13 | 9.49E−06 | 3.11E−04 |
| signaling | | | | | | | |
| pathway | | | | | | | |
| (P00036) | | | | | | | |

TABLE 6
Pathway analysis of genes in immune and other neoplasm cluster.

| | Homo | | | | | | |
|---|---|---|---|---|---|---|---|
| | sapiens - | Client Text | Client Text | | | | |
| PANTHER | REFLIST | Box Input | Box Input | | Fold | Raw | |
| Pathways | (20996) | (163) | (Expected) | Over/Under | Enrichment | P-value | FDR |
| Hypoxia | 32 | 6 | 0.29 | + | 20.83 | 1.01E−06 | 2.77E−05 |
| response via | | | | | | | |
| HIF | | | | | | | |
| activation | | | | | | | |
| (P00030) | | | | | | | |
| JAK/STAT | 17 | 3 | 0.15 | + | 19.6 | 7.12E−04 | 6.87E−03 |
| signaling | | | | | | | |
| pathway | | | | | | | |
| (P00038) | | | | | | | |
| Interleukin | 89 | 15 | 0.8 | + | 18.72 | 2.46E−14 | 1.34E−12 |
| signaling | | | | | | | |
| pathway | | | | | | | |
| (P00036) | | | | | | | |
| Insulin/IGF | 41 | 6 | 0.37 | + | 16.26 | 3.69E−06 | 7.56E−05 |
| pathway- | | | | | | | |
| protein | | | | | | | |
| kinase B | | | | | | | |
| signaling | | | | | | | |
| cascade | | | | | | | |
| (P00033) | | | | | | | |
| p53 pathway | 51 | 6 | 0.46 | + | 13.07 | 1.16E−05 | 1.90E−04 |
| feedback | | | | | | | |
| loops 2 | | | | | | | |
| (P04398) | | | | | | | |
| Toll receptor | 56 | 6 | 0.5 | + | 11.9 | 1.89E−05 | 2.82E−04 |
| signaling | | | | | | | |
| pathway | | | | | | | |
| (P00054) | | | | | | | |
| Interferon- | 29 | 3 | 0.26 | + | 11.49 | 2.87E−03 | 2.24E−02 |
| gamma | | | | | | | |
| signaling | | | | | | | |
| pathway | | | | | | | |
| (P00035) | | | | | | | |
| CCKR | 174 | 17 | 1.57 | + | 10.85 | 1.46E−12 | 5.97E−11 |
| signaling | | | | | | | |
| map | | | | | | | |
| (P06959) | | | | | | | |
| Insulin/IGF | 31 | 3 | 0.28 | + | 10.75 | 3.41E−03 | 2.54E−02 |
| pathway- | | | | | | | |
| mitogen | | | | | | | |
| activated | | | | | | | |
| protein | | | | | | | |
| kinase | | | | | | | |
| kinase/MAP | | | | | | | |
| kinase | | | | | | | |
| cascade | | | | | | | |
| (P00032) | | | | | | | |
| PI3 kinase | 53 | 5 | 0.48 | + | 10.48 | 1.67E−04 | 1.96E−03 |
| pathway | | | | | | | |
| (P00048) | | | | | | | |
| FAS | 33 | 3 | 0.3 | + | 10.1 | 4.02E−03 | 2.87E−02 |
| signaling | | | | | | | |
| pathway | | | | | | | |
| (P00020) | | | | | | | |
| Inflammation | 260 | 23 | 2.34 | + | 9.83 | 9.62E−16 | 7.89E−14 |
| mediated by | | | | | | | |
| chemokine | | | | | | | |
| and cytokine | | | | | | | |
| signaling | | | | | | | |
| pathway | | | | | | | |
| (P00031) | | | | | | | |
| VEGF | 69 | 6 | 0.62 | + | 9.66 | 5.63E−05 | 7.10E−04 |
| signaling | | | | | | | |
| pathway | | | | | | | |
| (P00056) | | | | | | | |
| T cell | 95 | 8 | 0.86 | + | 9.35 | 3.99E−06 | 7.27E−05 |
| activation | | | | | | | |
| (P00053) | | | | | | | |
| Apoptosis | 118 | 9 | 1.06 | + | 8.47 | 2.12E−06 | 4.97E−05 |
| signaling | | | | | | | |
| pathway | | | | | | | |
| (P00006) | | | | | | | |
| Ras Pathway | 74 | 5 | 0.67 | + | 7.51 | 7.08E−04 | 7.26E−03 |
| (P04393) | | | | | | | |
| Gonadotropin- | 230 | 14 | 2.07 | + | 6.76 | 4.34E−08 | 1.42E−06 |
| releasing | | | | | | | |
| hormone | | | | | | | |
| receptor | | | | | | | |
| pathway | | | | | | | |
| (P06664) | | | | | | | |
| EGF receptor | 134 | 8 | 1.21 | + | 6.63 | 4.19E−05 | 5.73E−04 |
| signaling | | | | | | | |
| pathway | | | | | | | |
| (P00018) | | | | | | | |
| p53 pathway | 87 | 5 | 0.78 | + | 6.38 | 1.41E−03 | 1.28E−02 |
| (P00059) | | | | | | | |
| B cell | 72 | 4 | 0.65 | + | 6.17 | 4.78E−03 | 3.26E−02 |
| activation | | | | | | | |
| (P00010) | | | | | | | |
| Angiogenesis | 173 | 8 | 1.56 | + | 5.14 | 2.27E−04 | 2.49E−03 |
| (P00005) | | | | | | | |
| FGF | 120 | 5 | 1.08 | + | 4.63 | 5.31E−03 | 3.48E−02 |
| signaling | | | | | | | |
| pathway | | | | | | | |
| (P00021) | | | | | | | |
| PDGF | 148 | 6 | 1.33 | + | 4.5 | 2.63E−03 | 2.16E−02 |
| signaling | | | | | | | |
| pathway | | | | | | | |
| (P00047) | | | | | | | |
| Alzheimer | 126 | 5 | 1.13 | + | 4.41 | 6.45E−03 | 4.07E−02 |
| disease- | | | | | | | |
| presenilin | | | | | | | |
| pathway | | | | | | | |
| (P00004) | | | | | | | |
| Integrin | 193 | 7 | 1.74 | + | 4.03 | 2.17E−03 | 1.87E−02 |
| signaling | | | | | | | |
| pathway | | | | | | | |
| (P00034) | | | | | | | |

Referring to panel A in FIG. 2 is a graph of unsupervised hierarchical clustering analysis of 1323 clinical RNA-seq profiles derived from a targeted RNA-sequencing expression of the aforementioned clinical cohort. There are three immunogenic clusters, namely, inflamed (n=439/1323; 33.18%), borderline (n=467/1323; 35.30%) and non-inflamed (n=417/1323; 31.52%). This 161 gene signature is over-represented by T & B cell activation pathways along with IFNg, chemokine, cytokine and interleukin pathways. Mean expression of these 161 genes constituting the immunogenic signature produces immunogenic score that leads to three distinct groups of strong (n=384/1323; 29.02%), moderate (n=354/1323; 26.76%) and weak (n=585/1323; 44.22%) immunogenicity. Referring to panel B in FIG. 2 are distributions of the immunogenic scores of the samples in each of the three sample clusters. Referring to panel C in FIG. 2 is a CD8 immunohistochemistry image of tumor with non-infiltrating T cells, panel D in FIG. 2 is a CD8 immunohistochemistry image of tumor with strongly infiltrating T cells, panel E in FIG. 2 is a CD8 immunohistochemistry image of tumor excluded from T cell tumor infiltration status classification, and panel F in FIG. 2 shows the distribution of immunogenic scores for tumors in the discovery cohort with non-infiltrating T cells, strongly infiltrating T cells, and those excluded from T cell tumor infiltration status classification.

In order to assess agreement of algorithmic immunogenic score with observed immune cell infiltration, the distribution of immunogenic score was analyzed within three major types of CD8 infiltration patterns estimated by IHC (infiltrating/strongly infiltrating, non-infiltrating, and excluded) (see, panels C-E in FIG. 2). As expected, the median immunogenic score of infiltrating/strongly infiltrating samples (n=493) was 54.85, whereas the median immunogenic score of noninfiltrating samples (n=403) was significantly lower (median=34.84; p=2.22E-16). Interestingly, excluded phenotype (n=26) of immune infiltration had a median immunogenic score similar to the strongly/moderately infiltrating phenotype (median=50.83; p=0.31), but significantly higher than the noninfiltrating pattern (p=0.00032) (see, panel F in FIG. 2).

### Results—TIS and Clinical Outcomes

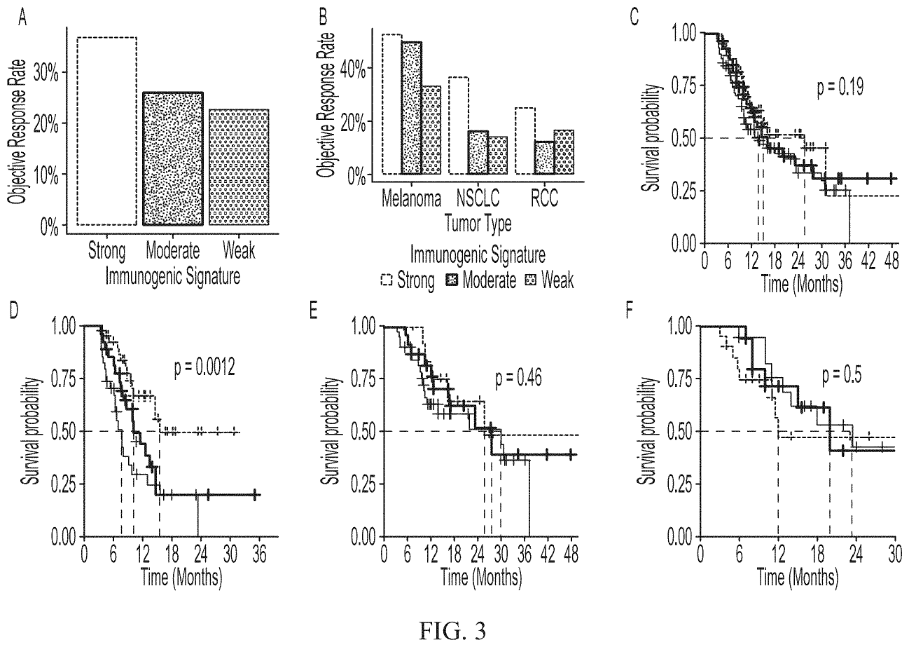

To assess the clinical utility of the immunogenic score, it was used to classify a previously published retrospective cohort of 242 samples (melanoma, NSCLC, and RCC) into strongly, moderately, and weakly immunogenic groups (see, panel A in FIG. 3). Strongly immunogenic tumors showed higher objective response rate (37%) compared to weakly immunogenic tumors (23%; p=0.06) to checkpoint inhibition in the pan-cancer retrospective cohort. Tumor type-specific analysis showed similar results in melanoma (53% vs. 33%; p=0.27), in NSCLC (36% vs. 14%; p=0.05), and RCC (25% vs 16%; p=0.8) (see, panel B in FIG. 3 and TABLE 7).

Referring to panel A in FIG. 3 is a graph of objective response rates observed in the retrospective cohort for each immunogenic score group, also known as the immunogenic signature, and panel B in FIG. 3 is a graph of objective response rate observed in each immunogenic score group for three disease types within the retrospective cohort. Panel C in FIG. 3 shows survival curves for each immunogenic signature group in the retrospective cohort, panel D in FIG. 3 shows a survival curve for each immunogenic signature group for lung cancer (NSCLC) cases in the retrospective cohort, panel E in FIG. 3 shows a survival curve for each immunogenic signature group for kidney cancer (KIRC) cases in the retrospective cohort, and panel F in FIG. 3 shows a survival curve for each immunogenic signature group for melanoma cases in the retrospective cohort.

TABLE 7
Objective response rates for immunogenic signature groups
in retrospective cohort for each disease type.

| Tumor Type | TIS Group | Responder | Non-responder | Total | ORR |
|---|---|---|---|---|---|
| Melanoma | Strong | 9 | 8 | 17 | 52.94% |
| | Moderate | 11 | 11 | 22 | 50.00% |
| | Weak | 13 | 26 | 39 | 33.33% |
| NSCLC | Strong | 16 | 28 | 44 | 36.36% |
| | Moderate | 5 | 26 | 31 | 16.13% |
| | Weak | 5 | 30 | 35 | 14.29% |
| RCC | Strong | 5 | 15 | 20 | 25.00% |
| | Moderate | 2 | 14 | 16 | 12.50% |
| | Weak | 3 | 15 | 18 | 16.67% |

The impact of immunogenic score on overall survival in the pan-cancer retrospective cohort was then investigated. Even though there was no significant difference in overall survival of strongly inflamed compared to weakly inflamed tumors (p=0.19), a clear separation of median survival between the two groups (25.6 months vs. 13.8 months) was observed (see, panel C in FIG. 3). The source of this difference was further investigated by performing tumor type-specific survival analysis, which showed that most of the survival advantage can be attributed to NSCLC cases (p=0.0012; 15.4 months vs. 7.63 months) (see, FIGS. 3D-3F and TABLE 8).

TABLE 8
Aggregate survival data for pan-cancer retrospective
cohort when grouped by TIS.

| | TIS | | | Median Survival | 95% | 95% |
|---|---|---|---|---|---|---|
| Tumor Type | Group | n | Events | (Months) | LCL | UCL |
| Pan-cancer | Strong | 81 | 28 | 25.6 | 14.5 | NA |
| | Moderate | 69 | 32 | 15 | 12.5 | NA |
| | Weak | 92 | 49 | 13.8 | 10 | 23.4 |
| Melanoma | Strong | 17 | 5 | 25.6 | 16.2 | NA |
| | Moderate | 22 | 9 | 27.6 | 16.6 | NA |
| | Weak | 39 | 18 | 29.9 | 11 | NA |
| NSCLC | Strong | 44 | 14 | 15.37 | 14.5 | NA |
| | Moderate | 31 | 17 | 10.13 | 8 | NA |
| | Weak | 35 | 23 | 7.63 | 6.3 | 13 |
| RCC | Strong | 20 | 9 | 12 | 11 | NA |
| | Moderate | 16 | 6 | 20 | 15 | NA |
| | Weak | 18 | 8 | 23.4 | 12.7 | NA |

### Results—TIS and Traditional Biomarkers

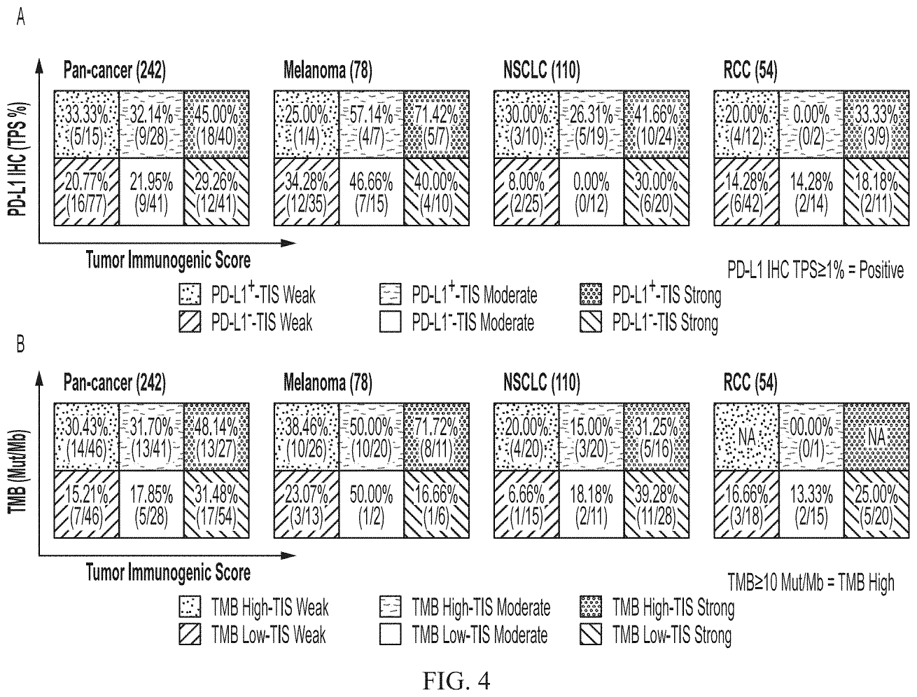

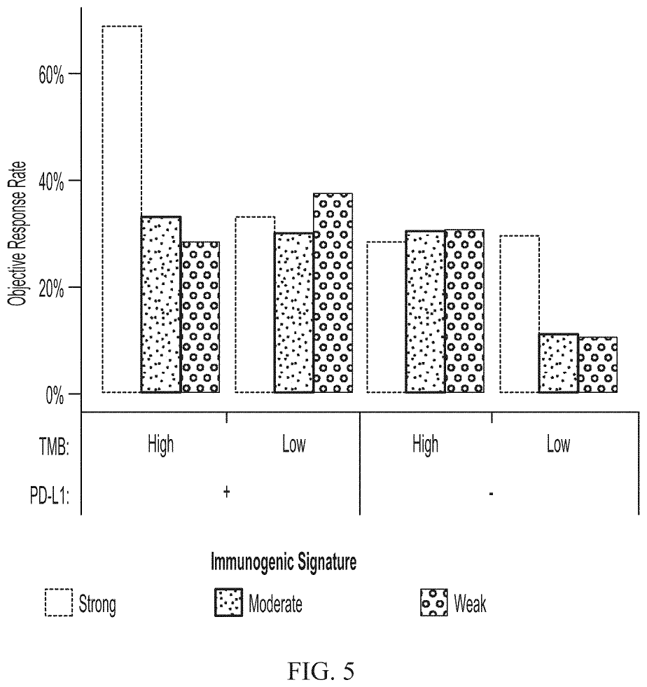

To further investigate the utility of TIS, the predictive capacity of TIS was studied in conjunction with traditional biomarkers for response to ICI therapy such as PD-L1 expression and high TMB. The combination of TIS and PD-L1 shows an additive effect on objective response rate to ICI therapy in the retrospective cohort, as shown in panel A in FIG. 4. A similar effect was observed for TMB, as shown in panel B in FIG. 3. In general, PD-L1+, strongly immunogenic patients had the highest clinical response rate for all three cancer types (excluding single-sample groups), and PD-L1−, weakly immunogenic patients had the lowest response rate (or in the case of melanoma, the second-lowest). Interestingly, PD-L1 and TMB in combination did not show a similar effect (see FIG. 5). In melanoma, TMB high, strongly inflamed patients had a response rate of 72.73%, while TMB low, strongly inflamed patients had a response rate of 16.67%.

Referring to panel A in FIG. 4 are objective response rates for each subgroup when TIS is used in conjunction with PD-L1 status, separated by disease type. Referring to panel B in FIG. 4 are objective response rates for each subgroup when TIS is used in conjunction with TMB status, separated by disease type.

Combining TIS with PD-L1 and TMB status for all cancer types, the prediction of objective response becomes even more robust, as shown in FIG. 5 (showing clinical response rates for each subgroup in the retrospective cohort when TIS is used in conjunction with TMB and PD-L1 IHC). A significantly higher [p=0.0001] objective response rate of 69.23% was observed for PD-L1 positive, TMB high, strongly inflamed tumors, compared to an objective response rate of only 10.53% for PD-L1 negative, non-TMB high, weakly inflamed tumors.

### Results—TIS and Cell Proliferation

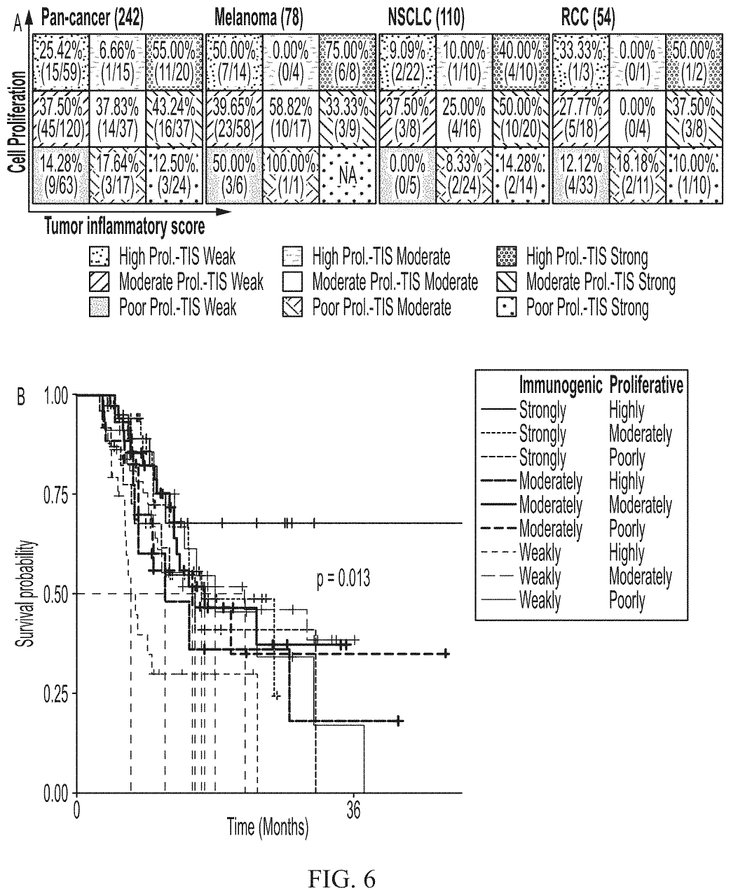

In order to gain more comprehensive insight into the tumor microenvironment and its effect on immunotherapy response, an understanding of both immune and neoplastic influences is required. To achieve this, TIS was combined with a previously published emerging biomarker of cell proliferation. Combining TIS groups with cell proliferation classes of highly, moderately, and poorly proliferative tumors significantly improves objective response separation, where highly proliferative, inflamed tumors [55%] have significantly higher objective response to ICI therapy than poorly proliferative, non-inflamed tumors [14.28%; p=0.0006]. See, panel A in FIG. 6. Tumor type-specific analysis could not be performed due to small sample sizes within each subgroup.

Supporting evidence was observed in significant survival differences between different combinations of TIS and cell proliferation [p=0.013], as shown in panel B in FIG. 6. Importantly, it is noted that strongly inflamed and highly [median=not achieved; p=0.025] or moderately [median =16.2 months; p=0.025] proliferative tumors had significantly better survival compared to weakly inflamed, highly proliferative tumors [median=7.03 months]. See TABLES 9 and 10. This data suggests that both T cell proliferation and tumor cell proliferation contribute to the signal in highly inflamed and highly proliferative tumors, whereas only tumor cell proliferation appears to contribute to the measurement of highly proliferative, weakly inflamed tumors. Therefore, combining both neoplastic and immune influences as described above could facilitate a more comprehensive understanding of the tumor immune microenvironment and likelihood of response to ICIs.

Referring to panel A in FIG. 6 are clinical response rates for each subgroup in the retrospective cohort when TIS is used in conjunction with cell proliferation score classification.

Panel B in FIG. 6 shows Kaplan Meier survival curves of combined TIS and cell proliferation status for 242 ICI treated retrospective cohort.

TABLE 9
Aggregate survival data for pan cancer retrospective
cohort when grouped by TIS and cell proliferation.

| | Cell | | | Median Survival | 95% | 95% |
|---|---|---|---|---|---|---|
| TIS | Proliferation | n | Events | (Months) | LCL | UCL |
| Strong | Highly | 20 | 5 | NA | 11.5 | NA |
| | Moderately | 37 | 12 | 16.2 | 12.03 | NA |
| | Poorly | 24 | 11 | 15.37 | 11 | NA |
| Moderate | Highly | 15 | 8 | 11.47 | 7.5 | NA |
| | Moderately | 37 | 15 | 16.63 | 12.63 | NA |
| | Poorly | 17 | 9 | 15 | 9.83 | NA |
| Weak | Highly | 24 | 16 | 7.03 | 6.3 | NA |
| | Moderately | 46 | 21 | 13.77 | 10.5 | NA |
| | Poorly | 22 | 12 | 18 | 12.7 | NA |

TABLE 10A
Pairwise comparison p-values for survival of pan-cancer retrospective
cohort when grouped by TIS and cell proliferation.

| | | Strongly Immunogenic | | | Moderately Immunogenic | | |
|---|---|---|---|---|---|---|---|
| TIS | Proliferation | Highly | Moderately | Poorly | Highly | Moderately | Poorly |
| Strongly Immunogenic | Highly | — | — | — | — | — | — |
| | Moderately | 0.62 | — | — | — | — | — |
| | Poorly | 0.378 | 0.701 | — | — | — | — |
| Moderately Immunogenic | Highly | 0.359 | 0.701 | 0.997 | — | — | — |
| | Moderately | 0.62 | 0.997 | 0.62 | 0.62 | — | — |
| | Poorly | 0.378 | 0.732 | 0.876 | 0.825 | 0.732 | — |
| Weakly Immunogenic | Highly | 0.025 | 0.025 | 0.359 | 0.359 | 0.04 | 0.359 |
| | Moderately | 0.378 | 0.825 | 0.826 | 0.781 | 0.825 | 0.908 |
| | Poorly | 0.378 | 0.997 | 0.732 | 0.825 | 0.97 | 0.97 |

TABLE 10B
Pairwise comparison p-values for survival of pan-cancer retrospective
cohort when grouped by TIS and cell proliferation.

| | | Weakly Immunogenic | | |
|---|---|---|---|---|
| TIS | Proliferation | Highly | Moderately | Poorly |
| Strongly | Highly | — | — | — |
| Immunogenic | Moderately | — | — | — |
| | Poorly | — | — | — |
| Moderately | Highly | — | — | — |
| Immunogenic | Moderately | — | — | — |
| | Poorly | — | — | — |
| Weakly | Highly | — | — | — |
| Immunogenic | Moderately | 0.09 | — | — |
| | Poorly | 0.09 | 0.97 | — |

### Discussion

Even though PD-L1 tumor proportion score by immunohistochemistry and Tumor Mutational Burden are among the most utilized biomarkers to ICI treatment decision making, the complexity of the antitumor host immune response cannot be fully explained by a single biomarker of immune or neoplastic mechanism. TMB is known to be correlated to response to ICI in multiple disease types however when evaluated for combination therapy there was no difference in median TMB for responders versus non-responders. Since TMB does not directly represent the neoantigen load comprised of immunogenic neopeptides, it may only lead to limited understanding of the T-IME being assessed. Similarly, PD-L1 by IHC was only found to be predictive in 28.9% of cases across 45 FDA drug approvals for ICI across 15 tumor types. This results in the need to investigate multiplex biomarkers, including tumor immunogenic signature, that are more comprehensive in deciphering the state of the tumor immune microenvironments primed for ICI response.

For a more comprehensive treatment decision a robust measurement of the host immune response is required. In this example is shown the discovery of comprehensive RNA-seq gene expression-based tumor immunogenic signature TIS that complements both traditional and emerging biomarkers of ICI response in solid tumors. Immunogenic signature was derived from a pan-cancer cohort of real-world clinical FFPE tumors to broadly describe immunogenic state of the tumor microenvironment as strongly, moderately and weakly inflamed. TIS score was highly correlated to the TIL infiltration pattern observed in the tumor samples. TIS also differentiated patients with higher response and improved survival in NSCLC. TIS score also complemented traditional biomarkers where, as expected PD-L1⁺ tumors that were strongly inflamed had a very high response (45%; 18/40). Interestingly, TIS was able to identify a subpopulation of PD-L1 negative tumors with strongly inflamed phenotype with response to ICI up to 29% (12/41). Similarly, TIS score complements TMB where TMB high tumors that are strongly inflamed have response rate of 48% (13/17), but was also able to identify non-TMB high, strongly inflamed cases that have response rate of 31% (17/54). Specifically focusing on NSCLC which is the largest population of the discovery cohort, it was observed that the clinical utility of TIS in this disease type. After conducting a retrospective analysis of 110 NSCLC samples using the clinically recommended immune checkpoint biomarkers of PD-L1 and TMB by next generation sequencing, a substantial subpopulation was identified of PD-L1−, TMB- patients (24%; n=26) of which 46% presented an inflamed TME as measured by TIS. These PD-L1−, TMB low, TIS inflamed patients had ORR of 42% whereas none of the PD-L1−, TMB low and moderately or weakly inflamed tumors responded to ICI (see TABLE 12). As such, the TIS serves as a novel method to identify a substantial cohort of NSCLC patients who would benefit from ICI that would not be identified by current clinical protocols.

TABLE 11
Objective response rates for pan-cancer retrospective cohort subdivided
by PD-L1 status, TMB status, cell proliferation classification, and TIS.

| | | | | | | | Objective |
|---|---|---|---|---|---|---|---|
| PD-L1 | TMB | Cell | TIS | | Non- | | Response |
| Status | Status | Proliferation | Signature | Responder | responder | Total | Rate |
| Positive | TMB | Highly Proliferative | Strong | 5 | 2 | 7 | 71.43% |
| | High | | Moderate | 1 | 6 | 7 | 14.29% |
| | | | Weak | 0 | 1 | 1 | 0.00% |
| | | Moderately | Strong | 4 | 2 | 6 | 66.67% |
| | | Proliferative | Moderate | 5 | 5 | 10 | 50.00% |
| | | | Weak | 2 | 4 | 6 | 33.33% |
| | | Poorly Proliferative | Strong | 0 | 0 | 0 | NA |
| | | | Moderate | | 1 | 1 | 0.00% |
| | | | Weak | 0 | 0 | 0 | NA |
| | TMB | Highly Proliferative | Strong | 2 | 5 | 7 | 28.57% |
| | Low | | Moderate | 0 | 1 | 1 | 0.00% |
| | | | Weak | 1 | 5 | 6 | 16.67% |
| | | Moderately | Strong | 5 | 8 | 13 | 38.46% |
| | | Proliferative | Moderate | 3 | 4 | 7 | 42.86% |
| | | | Weak | 1 | 0 | 1 | 100.00% |
| | | Poorly Proliferative | Strong | 2 | 5 | 7 | 28.57% |
| | | | Moderate | 0 | 2 | 2 | 0.00% |
| | | | Weak | 1 | 0 | 1 | 100.00% |
| Negative | TMB | Highly Proliferative | Strong | 2 | 2 | 4 | 50.00% |
| | High | | Moderate | 0 | 5 | 5 | 0.00% |
| | | | Weak | 2 | 11 | 13 | 15.38% |
| | | Moderately | Strong | 2 | 4 | 6 | 33.33% |
| | | Proliferative | Moderate | 6 | 11 | 17 | 35.29% |
| | | | Weak | 9 | 15 | 24 | 37.50% |
| | | Poorly Proliferative | Strong | 0 | 4 | 4 | 0.00% |
| | | | Moderate | 1 | 0 | 1 | 100.00% |
| | | | Weak | 1 | 1 | 2 | 50.00% |
| | TMB | Highly Proliferative | Strong | 2 | 0 | 2 | 100.00% |
| | Low | | Moderate | 0 | 2 | 2 | 0.00% |
| | | | Weak | 0 | 4 | 4 | 0.00% |
| | | Moderately | Strong | 5 | 7 | 12 | 41.67% |
| | | Proliferative | Moderate | 0 | 3 | 3 | 0.00% |
| | | | Weak | 3 | 12 | 15 | 20.00% |
| | | Poorly Proliferative | Strong | 1 | 12 | 13 | 7.69% |
| | | | Moderate | 2 | 11 | 13 | 15.38% |
| | | | Weak | 1 | 18 | 19 | 5.26% |

TABLE 12
Objective response rates for a subpopulation of PD-L1 - and TMB low
(n = 26) of the NSCLC retrospective cohort for three TIS groups.

| TIS Score | Responder | Non-responder | Total | Objective Response Rate |
|---|---|---|---|---|
| Strong | 5 | 7 | 12 | 41.67% |
| Moderate | 0 | 4 | 4 | 0.00% |
| Weak | 0 | 10 | 10 | 0.00% |

The TIS was then combined with cell proliferation which is an emerging biomarker for resistance to ICI therapy in NSCLC and RCC. As previously published moderately proliferative tumors had significantly higher response to ICI as compared to poorly or highly proliferative tumors regardless of immunogenicity, except in the case of highly inflamed tumors. Highly inflamed and highly proliferative tumors had the highest response rate in the pan-cancer retrospective cohort. This led to the hypothesis that a TIS score represents the host immune response and cell proliferation represents the overall proliferative potential of the entire TME. In case of strongly inflamed and highly proliferative tumors, the cell proliferation signal can be attributed to antigen stimulated T cell proliferation as well as tumor cell proliferation. This TME is uniquely primed for response to ICI therapy. However, weakly inflamed tumors may not contribute to cell proliferation signal via antigen stimulated T cell proliferation. Therefore, most of the cell proliferation signal may be attributed to tumor proliferation making the TME resistance to ICI therapy due to lack of underlying host immune response. Combining the TIS score and cell proliferation with traditional biomarkers of PD-L1 and TMB support this merger. Here, in the pan-cancer retrospective cohort it was possible to identify PD-L1 TMB low patients that had very high response rate for highly proliferative, strongly inflamed tumors (100%; 2/2) and moderately proliferative, strongly inflamed tumors (42%; 5/12). As such, the TIS score in conjunction with traditional and emerging biomarkers of ICI response and resistance provides a comprehensive understanding of the underlying state of immune and neoplastic influences that contribute to the success of failure of ICI therapy.

Although the example was not based on controlled trial samples, the immunogenic score was derived from a large cohort of real world clinical FFPE samples spanning multiple solid tumor types. One future avenue of research is larger subgroup sample sizes to perform sufficiently powered analysis when combines multiple biomarkers. This led to the study of a pooled analysis on the retrospective cohort while not being able to separate the dataset further by ICI treatment agent. Additionally, due to low sample size for RCC and Melanoma retrospective cohort also limits the analysis one could perform on a subgroup level. Considering these limitations, it is believed that further studies are warranted to tease out some tumor type and treatment type specific effects of immunogenic score alone and in conjunction with other biomarkers. However, it is believed this large-scale assessment of clinical grade cohort will lead to further hypothesis testing of integration of immune and neoplastic signals in the tumor immune microenvironment.

### CONCLUSIONS

In summary, the example demonstrates that the comprehensive tumor immunogenic signature not only describes the underlying host immune response but also integrates with biomarkers of ICI response such as PD-L1 and TMB along with biomarkers of resistance to ICI such as cell proliferation. TIS score alone as well as in combination with these biomarkers can identify patient subpopulations that may be resistance to ICI therapy but more importantly select patients that may have not been identified to for response to ICI by traditional clinical biomarkers.

While embodiments of the present invention have been particularly shown and described with reference to certain exemplary embodiments, it will be understood by one skilled in the art that various changes in detail may be effected therein without departing from the spirit and scope of the invention as defined by claims that can be supported by the written description and drawings. Further, where exemplary embodiments are described with reference to a certain number of elements it will be understood that the exemplary embodiments can be practiced utilizing either less than or more than the certain number of elements.

What is claimed is:

1. A method for characterizing response of a tumor to immunotherapy, comprising:
obtaining tissue from the tumor;
generating, from the obtained tissue, an immune gene expression dataset comprising gene expression data for a plurality of immune genes;
calculating, from the immune gene expression dataset, an immunogenic signature score;
identifying, based on the calculated immunogenic signature score, the tumor as strongly immunogenic, moderately immunogenic, or weakly immunogenic; and
predicting, based on the identification of the tumor as strongly immunogenic, moderately immunogenic, or weakly immunogenic, the response of the tumor to immunotherapy.

2. The method of claim 1, wherein the plurality of immune genes comprises at least the 161 genes of Table 4.

3. The method of claim 1, wherein the plurality of immune genes comprises only the 161 genes of Table 4.

4. The method of claim 1, wherein the plurality of immune genes comprises a subset of the 161 genes of Table 4.

5. The method of claim 1, wherein the immunogenic signature score comprises a mean expression rank for the gene expression data for the plurality of immune genes.

6. The method of claim 1, further comprising:
generating, from the obtained tissue, a cell proliferation gene expression dataset comprising gene expression data for a plurality of cell proliferation genes;
calculating, from the cell proliferation gene expression dataset, a cell proliferation score; and
identifying, based on the calculated cell proliferation score, the tumor as highly proliferative, moderately proliferative, or poorly proliferative;
wherein predicting the response of the tumor to immunotherapy is further based on the identification of the tumor as highly proliferative, moderately proliferative, or poorly proliferative.

7. The method of claim 1, further comprising:
generating, from the obtained tissue, a PD-L1 expression profile;
wherein predicting the response of the tumor to immunotherapy is further based on the generated PD-L1 expression profile.

8. The method of claim 1, further comprising:
generating, from the obtained tissue, a tumor mutational burden (TMB) profile, wherein the TMB profile comprises mutational burden information about a plurality of genes generated from DNA sequencing data;
wherein predicting the response of the tumor to immunotherapy is further based on the generated TMB profile.

9. The method of claim 1, further comprising the step of determining, using the predicted response of the tumor to immune checkpoint blockade therapy, a therapy for the tumor.

10. The method of claim 1, wherein the tumor as is identified as strongly immunogenic when the calculated immunogenic signature score (IS) is equal to and/or greater than [Median IS]_{Borderline}+2×[Std. Dev. IS]_{Borderline}, wherein [Median IS]_{Borderline} is a median determined for a set of immunogenic signature scores calculated for a plurality of patients categorized as borderline inflamed, and [Std. Dev. IS]_{Borderline} is one standard deviation of the set of immunogenic signature scores calculated for the plurality of patients categorized as borderline inflamed.

11. The method of claim 10, wherein the tumor as is identified as weakly immunogenic when the calculated immunogenic signature score (IS) is equal to and/or less than [Median IS]_{Noninflamed}+2×[Std. Dev. IS]_{Noninflamed}, wherein [Median IS]_{Noninflamed} is a median determined for a set of immunogenic signature scores calculated for a plurality of patients categorized as noninflamed, and [Std. Dev. IS]_{Noninflamed} is one standard deviation of the set of immunogenic signature scores calculated for the plurality of patients categorized as noninflamed.

12. The method of claim 11, wherein the tumor is identified as moderately immunogenic when the calculated immunogenic signature score (IS) determined to be less than a strongly immunogenic score and greater than a weakly immunogenic score.

13. A method for characterizing response of a tumor to immunotherapy, comprising:
obtaining tissue from the tumor;
generating, from the obtained tissue: (1) an immune gene expression dataset comprising gene expression data for a plurality of immune genes; (2) a PD-L1 expression profile; and (3) a tumor mutational burden (TMB) profile, wherein the TMB profile comprises mutational burden information about a plurality of genes generated from DNA sequencing data;
calculating, from the immune gene expression dataset, an immunogenic signature score;
identifying, based on the calculated immunogenic signature score, the tumor as strongly immunogenic, moderately immunogenic, or weakly immunogenic; and
predicting, based on: (1) the identification of the tumor as strongly immunogenic, moderately immunogenic, or weakly immunogenic; (2) the generated PD-L1 expression profile; and (3) the generated TMB profile, the response of the tumor to immunotherapy.

14. The method of claim 13, further comprising:
generating, from the obtained tissue, a cell proliferation gene expression dataset comprising gene expression data for a plurality of cell proliferation genes;
calculating, from the cell proliferation gene expression dataset, a cell proliferation score; and
identifying, based on the calculated cell proliferation score, the tumor as highly proliferative, moderately proliferative, or poorly proliferative;
wherein predicting the response of the tumor to immunotherapy is further based on the identification of the tumor as highly proliferative, moderately proliferative, or poorly proliferative.

15. The method of claim 13, wherein the plurality of immune genes comprises at least the 161 genes of Table 4.

16. The method of claim 13, wherein the plurality of immune genes comprises only the 161 genes of Table 4.

17. The method of claim 13, wherein the plurality of immune genes comprises a subset of the 161 genes of Table 4.

18. The method of claim 13, wherein the immunogenic signature score comprises a mean expression rank for the gene expression data for the plurality of immune genes.

19. The method of claim 1, wherein the tumor as is identified as strongly immunogenic when the calculated immunogenic signature score (IS) is equal to and/or greater than [Median IS]_{Borderline}+2×[Std. Dev. IS]_{Borderline}, wherein [Median IS]_{Borderline} is a median determined for a set of immunogenic signature scores calculated for a plurality of patients categorized as borderline inflamed, and [Std. Dev. IS]_{Borderline} is one standard deviation of the set of immunogenic signature scores calculated for the plurality of patients categorized as borderline inflamed.

20. The method of claim 19, wherein the tumor as is identified as weakly immunogenic when the calculated immunogenic signature score (IS) is equal to and/or less than [Median IS]_{Noninflamed}²×[Std. Dev. IS]_{Noninflamed} wherein [Median IS]_{Noninflamed} is a median determined for a set of immunogenic signature scores calculated for a plurality of patients categorized as noninflamed, and [Std. Dev. IS]_{Noninflamed} is one standard deviation of the set of immunogenic signature scores calculated for the plurality of patients categorized as noninflamed.
